(19) United States
(12) Patent Application Publication
(10) Pub. No.: US 2021/0338650 A1
(43) Pub. Date: Nov. 4, 2021

(54) CXCL10 Inhibitors

(71) Applicant: LAPKO INC, dba AFECTA PHARMACEUTICALS, Las Vegas, NV (US)

(72) Inventor: Bruce KOVACS, Long Beach, CA (US)

(73) Assignee: AFECTA PHARMACEUTICALS, INC., Irvine, CA (US)

(21) Appl. No.: 17/242,867

(22) Filed: Apr. 28, 2021

Related U.S. Application Data
(60) Provisional application No. 63/017,137, filed on Apr. 29, 2020.

(51) Int. Cl. A61K 31/4439; A61P 27/02; A61P 17/00

(52) U.S. Cl. CPC A61K 31/4439; A61P 17/00; A61P 27/02

(57) ABSTRACT

Provided herein are compounds and compositions effective for inhibiting CXCL10 gene expression, production, and secretion in mammalian cells, tissues and organs, as well as ameliorating its biological activity, along with a method for treatment of one or more disorders associated with an increase in CXCL10 gene expression, production, and secretion.

This Nonprovisional application claims priority under 35 U.S.C. § 119(e) on U.S. Provisional Application No. 63/017,137 filed on Apr. 29, 2020, the contents of which are hereby incorporated by reference in its entirety.

### FIELD OF THE INVENTION

This invention pertains to inhibitors of the production of CXCL10 and amelioration of its biological activity on mammalian cells and tissues. The invention provides compounds and methods useful for inhibition of the biological effects of CXCL10 on mammalian cells and for the treatment of human diseases associated with increased levels of CXCL10.

### BACKGROUND OF THE INVENTION

CXCL10 is a small molecular weight inducible chemokine that has been found to be present in high levels in a variety of human diseases. CXCL10 is not normally produced by mammalian cells and tissues however. Certain types of normal mammalian and human cells, such as endothelial, epithelial, fibroblast, keratinocyte, smooth muscle, mesangial, astrocyte, mononuclear, and microglial cells, can be induced to produce and secrete high levels of CXCL10 by stimulation with various substances associated with infectious agents, such as lipopolysaccharide (LPS). In addition, a variety of stress conditions and substances that mimic bacterial, viral and fungal components, such as Poly (I:C), induce production of CXCL10 in normal cells. In certain diseases, immune, metabolic, neoplastic disorders and cancers CXCL10 is produced in relatively large amounts and is present in high levels in the blood and other tissue fluids in humans suffering from these disorders.

CXCL10 binds to and transduces signals through its receptor CXCR3. This receptor is present on many types of human cells such as, but not limited to: leukocytes, peripheral blood mononuclear cells (PBMCs), fibroblasts, vascular smooth muscle cells, endothelial cells, and astrocytes (Vazrinejad R, et al. Neuroimmunomodulation 2014; 21:322-330). The variety of cells that express CXCR3 and mediate CXCL10 biological effects implicates CXCL10 in many human disorders, such as: arthritis, asthma, cancer, dermatitis, endometriosis, lichen planus, psoriasis, Sjogren syndrome, uveitis, and others. However, CXCL10 may also produce biological effects through non-CXCR3 dependent mechanisms.

Increased CXCL10 production by human endothelial, epithelial, fibroblasts, keratinocytes, smooth muscle, mesangial, astrocytic, monocytic, microglial, or retinal cells and the like promotes disease initiation and/or progression, subsequent tissue destruction, and loss of function in various human tissues such as, but not limited to, the dermis, epidermis, ocular uvea, macula, retina, pulmonary bronchial, and urogenital epithelium. In addition, inherited genetic variants in the CXCL10 gene region are associated with several diseases and promote susceptibility to diseases. Therefore, CXCL10 is an important therapeutic target to treat various diseases such as those referenced herein. There are no approved drugs on the market that reduce cellular production of CXCL10. Thus, there is a need for chemical compounds that inhibit the production of CXCL10 by mammalian cells.

### SUMMARY OF THE INVENTION

As described herein the inventor has discovered that compounds embodied by Formula I inhibit the gene expression, production and secretion of the bioactive protein CXCL10 from mammalian cells, tissues and organs. In certain embodiments this disclosure provides compounds that are therefore capable of inhibiting the subsequent biologic effects elicited by CXCL10 on mammalian cells, tissues and organs and methods of treatment, prevention, inhibition or amelioration of one or more diseases associated with CXCL10 using the compound and compositions disclosed herein.

According to one embodiment of the present invention, there is provided inhibition of the production of CXCL10 by mammalian cells, tissues and organs comprising the topical application, either alone or in combination with other bioactive substances, of at least one compound according to Formula 1 below:

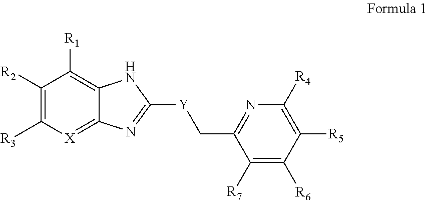

wherein:

X is selected from CH or N;

Y is selected from CH₂, CHOH, C(optionally substituted C₁ to C₈ straight chain or branched chain alkyl)OH, C═O, NH, N-optionally substituted C₁ to C₈ straight chain or branched chain alkyl, NCO-optionally substituted C₁ to C₈ straight chain or branched chain alkyl, S, S═O, SO₂;

R₁, R₂, R₃, R₄, R₅, R₆, and R₇ are independently selected from: H; OH; F; Cl; Br; I; (halogen)alkyl, optionally substituted C₁ to C₈ straight chain or branched chain alkyl; optionally substituted C₁ to C₈ cycloalkyl; heterocycloalkyl; alkylheterocycloalkyl; optionally substituted C₁ to C₈ alkenyl; optionally substituted C₁ to C₈ alkynyl; optionally substituted aryl; optionally substituted alkylaryl; optionally substituted heteroaryl; optionally substituted alkylheteroaryl; O-alkyl; O-optionally substituted alkyl, O-cycloalkyl; O-alkylcycloalkyl; O-aryl; O-optionally substituted aryl; alkyl-O-aryl; alkyl-O-optionally substituted aryl; C(O)-aryl; C(O)-optionally substituted aryl; CH₂C(O)-aryl; CH₂C(O)-optionally substituted aryl; O-(halogen)alkyl, or adjacent substituents R₁ and R₂, R₂ and R₃, R₄ and R₅, R₅ and R₆, R₆ and R₇, may form a saturated or unsaturated 5 membered or 6-membered or 7 membered carbocyclic or heterocyclic ring, and optionally substituted alkenyl, if present, may have one or more double bond and each double bond may independently be cis or trans, E or Z, a cis/trans mixture or an E/Z mixture, wherein if an asymmetric center is present or asymmetric centers are present the compound may be in the form of a racemic mixture, a single enantiomer, a diastereoisomeric mixture, an enantiomeric diastereomer, a meso compound, a pure epimer, or a mixture of epimers thereof, and a hydrogen, several hydrogens or all hydrogens may be replaced with deuterium or the compound is a pharmaceutically acceptable salt, ester or prodrug form thereof.

An additional embodiment of the invention is the inhibition of the biological effects of CXCL10 on mammalian cells such as, but not limited to, epithelial cells, mononuclear cells, as well as human tissues by application to these cells or tissues with a compound or compounds according to Formula 1, either alone or in combination with other bioactive substances, a physiological pH.

A further embodiment of the invention is the use of a compound as defined above in the manufacture of a medicament for the treatment of a mammal at risk for or having at least one disease or disorder associated with elevated CXCL10 levels. Non-limiting examples of such disorders are: skin disorders and/or diseases; urticarial conditions; respiratory ailments; airway and pulmonary conditions; ocular disorders; genito-urinary disorders; neurologic disorders, neoplastic disorders, cancers, infection-based diseases; fibrotic disorders, and trauma and tissue injury-based conditions.

### BRIEF DESCRIPTIONS OF THE DRAWINGS

FIG. 1a-b: Decreased gene expression and cellular production of CXCL10 by human keratinocyte cells after treatment with compounds of Formula 1. Cells were stimulated with Poly (I:C) and the relative gene expression of CXCL10 (FIG. 1b) and induced production and secretion of CXCL10 (FIG. 1b) were measured relative to controls cells not treated with the compounds of Formula 1.

FIG. 2a-b: Decreased gene expression and cellular production of CXCL10 by human bronchial epithelial cells after treatment with compounds of Formula 1. Cells were stimulated with Interferon gamma (IFN-γ) and the relative gene expression of CXCL10 (FIG. 2a) and induced production and secretion of CXCL10 (FIG. 2b) were measured relative to controls cells not treated with the compounds of Formula 1.

FIG. 3a-b: Decreased gene expression and cellular production of CXCL10 by human peripheral blood mononuclear cells after treatment with compounds of Formula 1. Cells were stimulated with Lipopolysaccharide (LPS) and the relative gene expression of CXCL10 (FIG. 3a) and induced production and secretion of CXCL10 (FIG. 3b) were measured relative to controls cells not treated with the compounds of Formula 1.

FIG. 4a-b: Decreased gene expression and production of CXCL10 by reconstructed full thickness human epithelium after treatment with compounds of Formula 1. Cells were stimulated with Interferon gamma (IFN-γ) plus Tumor Necrosis Factor alpha (TNF-α) and the relative gene expression of CXCL10 (FIG. 4a) and induced production and secretion of CXCL10 (FIG. 4b) were measured relative to controls tissues not treated with the compounds of Formula 1.

FIG. 5a-b: Reduction in CXCL10 levels in retina tissue and reduction in retinal vascular permeability in diabetic rats treated with compounds according to Formula 1. FIG. 5a presents a graph of CXCL10 protein levels as measured by ELISA. FIG. 5b presents a graph of retinal vascular permeability factor as a measurement of [concentration of Evans Blue dye in retina]/[concentration of Evans Blue dye in plasma×circulation time].

FIG. 6: Reduction in dermatitis/eczema lesions in human subjects treated with compound according to Formula 1. Subjective score based on Investigator's Global Assessment (IGA).

FIG. 7: Reduction in Topical Endoscopic Fundal Imaging (TEFI) score of mice with experimental autoimmune uveoretinitis (EAU) treated with a compound according to Formula 1 verses vehicle control alone.

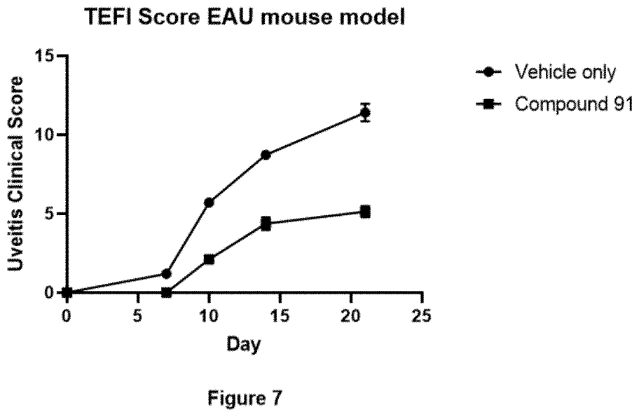

### DETAILED DESCRIPTION OF INVENTION

Compounds embodied by Formula I inhibit the gene expression, production and secretion of the bioactive protein CXCL10 from mammalian cells, tissues and organs. In certain embodiments this disclosure provides compounds that are therefore capable of inhibiting the subsequent biologic effects elicited by CXCL10 on mammalian cells, tissues and organs and methods of treatment, prevention, inhibition, or amelioration of one or more diseases associated with CXCL10 using the compounds and compositions disclosed herein.

Compounds embodied by Formula I may have one or several asymmetric centers and therefore can exist in different stereoisomeric configurations. Consequently, the compounds of Formula I can occur as individual (pure) enantiomers, individual pure enantiomeric diastereomers as well as a mixture of enantiomers or diastereomers. The scope of the present invention includes both single enantiomers and mixtures thereof in all ratios. The scope of the present invention further includes all tautomeric forms (“tautomers”) of the compounds of Formula I, and all mixtures thereof in any ratio. It will be appreciated by one skilled in the art that a single compound may exhibit more than one type of isomerism.

The enantiomeric compounds of Formula I may be resolved into their pure enantiomers by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, specific stereoisomers maybe synthesized by using an optically active starting material, by asymmetric synthesis using optically active reagents, substrates, catalysts or solvents, or by converting one stereoisomer into the other by asymmetric transformation or inversion.

The compounds of the present invention may exist in unsolvated as well as a variety of solvated forms with pharmaceutically acceptable solvents such as water, ethanol, and the like. In general, the solvated forms are considered equivalent to the unsolvated forms for the purposes of the present invention. It should be understood that pharmaceutically acceptable solvents further includes isotopically substituted solvents such as D₂O, dimethyl sulfoxide-d6 and the like. The term ‘solvate’ is used herein to describe a complex comprising the compound of the invention and one or more pharmaceutically acceptable solvent molecules, including water. As such, all manner of hydrates of the compound are included by the term ‘solvate’. It is intended that the present invention embrace unsolvated forms, solvated forms and mixtures of solvated forms in any ratio.

The compounds of the present invention and/or its salts and/or solvate may exist as amorphous solids or may exist in one or more crystalline states, i.e. polymorphs. Polymorphs of the compound of Formula I are encompassed in the present invention and may be prepared by crystallization under a number of different conditions such as, for example, using different solvents or different solvent mixtures; crystallization at different temperatures; and using various modes of cooling ranging from very fast to very slow during crystallization. Polymorphs may also be obtained by heating or melting a compound of Formula I followed by gradual or fast cooling. The presence of polymorphs may be determined by solid NMR spectroscopy, IR spectroscopy, differential scanning calorimetry, powder x-ray diffraction or other techniques. It should be understood that all such crystalline and amorphous forms of the compound of Formula I, and its salts, solvates and prodrugs thereof are encompassed by the invention and the claims.

The present invention also includes all pharmaceutically acceptable isotopically-labeled variations of the compound of Formula I. Such isotopically-labeled variations are compounds having the same Formula and molecular formula as the compound of Formula I but wherein one or more atoms are replaced by atoms having an atomic mass or mass number different from the atomic mass or mass number usually found in nature. Examples of isotopes that may be incorporated into the compound of the present invention include isotopes of hydrogen, carbon, fluorine, nitrogen, and oxygen, such as 2H, 3H, 11C, 13C, 14C, 18F, 13N 15N 17O and 18O, respectively.

Certain isotopically labeled variations of the compound of the present invention such as, for example, those incorporating a radioactive isotope such as 3H and 14C, are useful in drug and/or substrate tissue distribution studies. Tritium, i.e. 3H, and carbon-14, i.e. 14C, are particularly preferred due their ease of preparation and detection. Further, substitution with heavier isotopes such as deuterium, i.e. 2H, can afford certain therapeutic advantages resulting from greater metabolic stability, for example, increased in vivo half-life or reduced dosage requirements, and hence may be preferred in some circumstances. Isotopically labeled compounds of Formula I of this invention and prodrugs thereof can generally be prepared by carrying out the procedures disclosed in the Schemes and/or in the Examples by substituting a readily available isotopically labeled reagent for a non-isotopically labeled reagent.

The compounds of Formula I may be administered as a prodrug. The term prodrug refers to a compound which is transformed in vivo to a compound of Formula I, or a pharmaceutically acceptable salt or solvate of the compound. The transformation may occur by various mechanisms, such as via hydrolysis in blood. A prodrug of the compound of Formula I may be formed in a conventional manner according to methods known in the art. A thorough discussion of prodrugs is provided by V. Stella in Pro-drugs as Novel Delivery Systems, Vol. 14 of the A.C.S. Symposium Series (Stella (1975)), and in Bioreversible Carriers in Drug Design (Roche (1987)), both of which are incorporated herein by reference.

“Alkyl” means a straight or branched chain, saturated hydrocarbon radical. By way of example, the hydrocarbon chain may have from one to twenty carbons, one to sixteen carbons, one to fourteen carbons, one to twelve carbons, one to ten carbons, one to eight carbons, one to six carbons, one to four carbons, etc. “Lower alkyl” may refer to alkyls having, e.g., one to six carbons, one to four carbons, etc. In certain examples, a straight chain alkyl may have from one to six carbon atoms and a branched alkyl three to six carbon atoms, e.g., methyl, ethyl, propyl, 2-propyl, butyl (including all isomeric forms), pentyl (including all isomeric forms), and the like. “Me” means methyl, “Et” means ethyl, and “iPr” means isopropyl. Alkyl may be optionally substituted, e.g., optionally substituted with oxygen, silicon, sulphur or optionally substituted with OH, O-alkyl, SH, S-alkyl, NH2, NH-alkyl. In another example, alkyl may be C1 to C12 straight chain or branched chain alkyl optionally substituted with oxygen, silicon, sulphur or optionally substituted with OH, O-alkyl, SH, S-alkyl, NH2, NH-alkyl.

“Alkylene” means a divalent alkyl, with alkyl as defined above.

“Aryl” means a monocyclic or bicyclic aromatic hydrocarbon radical, e.g., having from of 6 to 20 or 6 to 10 ring atoms e.g., phenyl or naphthyl. Aryl may be optionally substituted, e.g., substituted phenyl or substituted naphthyl.

“Alkylaryl” means a (alkylene)-R radical where R is aryl as defined above. Alkylaryl may be optionally substituted. In certain examples, alkylaryl may be alkylphenyl, alkylsubstituted phenyl, alkylnaphthyl or alkylsubstituted naphthyl.

“Alkenyl” means a straight or branched chain, saturated hydrocarbon radical which contains a carbon-carbon double bond. By way of example, the hydrocarbon chain may have from two to twenty carbons, two to sixteen carbons, two to fourteen carbons, two to twelve carbons, two to ten carbons, two to eight carbons, two to six carbons, two to four carbons, etc. “Lower alkenyl” may refer to alkenyls having, e.g., two to six carbons, two to four carbons, etc. In certain examples, a straight chain alkenyl may have from two to six carbon atoms and a branched alkyl three to six carbon atoms, e.g., a vinyl group, an allyl group, butene (including all isomeric forms), pentene (including all isomeric forms), and the like. Alkenyl may be optionally substituted. In certain examples, alkenyl may be a C2 to C12 straight chain or branched chain hydrocarbon containing a carbon-carbon double bond, optionally substituted with oxygen, silicon or sulphur or optionally substituted with OH, O-alkyl, SH, S-alkyl, NH2 or NH-alkyl.

“Alkynyl” means a straight or branched chain, saturated hydrocarbon radical which contains a carbon-carbon triple bond. By way of example, the hydrocarbon chain may have from two to twenty carbons, two to sixteen carbons, two to fourteen carbons, two to twelve carbons, two to ten carbons, two to eight carbons, two to six carbons, two to four carbons, etc. “Lower alkynyl” may refer to alkynyls having, e.g., two to six carbons, two to four carbons, etc. In certain examples, a straight chain alkynyl may have from two to six carbon atoms and a branched alkyl three to six carbon atoms, e.g., an acetylene group, a propargyl group, butyne (including all isomeric forms), pentyne (including all isomeric forms), and the like. Alkynyl may be optionally substituted. In certain examples, alkynyl may be a C2 to C12 straight chain or branched chain hydrocarbon containing a carbon-carbon triple bond, optionally substituted with oxygen, silicon or sulphur or optionally substituted with OH, O-alkyl, SH, S-alkyl, NH2 or NH-alkyl.

“Cycloalkyl” means a cyclic saturated or partially saturated hydrocarbon radical (or an alicyclic radical). By way of example, the cycloalkyl may have from three to twenty carbon atoms, from three to sixteen carbon atoms, from three to fourteen carbon atoms, from three to twelve carbon atoms, from three to ten carbon atoms, from three to eight carbon atoms, from three to seven carbon atoms, from three to six carbon atoms, etc., wherein one or two carbon atoms may be replaced by an oxo group, e.g., admantanyl, cyclopropyl, cyclobutyl, cyclopentyl, cyclohexyl, cyclohexenyl, indanyl and the like.

“Alkylcycloalkyl” means a (alkylene)-R radical where R is cycloalkyl as defined above; e.g., cyclopropylmethyl, cyclobutylmethyl, cyclopentylethyl, or cyclohexylmethyl, and the like. In another example, alkylcycloalkyl has four to twelve carbon atoms, i.e., C4-C12 alkylcycloalkyl.

“O-alkyl” means an (oxygen)-R radical where R is alkyl as defined above. For example, O-alkyl may be an oxygen atom bonded to a C1 to C6 straight chain or branched chain alkyl.

“O-cycloalkyl” means an (oxygen)-R radical where R is cycloalkyl as defined above. For example, O-cycloalkyl is an oxygen atom bonded to a C3 to C7 cycloalkyl.

“O-alkylcycloalkyl” means an (oxygen)-R radical where R is alkylcycloalkyl as defined above. For example, O-cycloalkyl is an oxygen atom bonded to a C4 to C8 alkylcycloalkyl.

“Heterocyclyl” or “heterocycloalkyl” means a saturated or unsaturated monocyclic group, in which one or two ring atoms are heteroatom selected from N, O, or S, the remaining ring atoms being C. Heterocyclyl and heterocycloalkyl includes, e.g., where the heterocycle comprises one or two hetero atoms selected from O, S, or N, including a C2 to C6 heterocycloalkyl. The heterocyclyl ring is optionally fused to a (one) aryl or heteroaryl ring as defined herein. The heterocyclyl ring fused to monocyclic aryl or heteroaryl ring is also referred to in this Application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Alkylheterocycloalkyl” means an -(alkylene)-R radical where R is heterocyclyl ring as defined above e.g., tetraydrofuranylmethyl, piperazinylmethyl, morpholinylethyl, and the like. Alkylheterocycloalkyl also includes, e.g., where the heterocycle comprises one or two hetero atoms selected from O, S, or N and has three to eleven carbon atoms, i.e., C3 to C11 alkylheterocycloalkyl, and includes when N is present in the heterocyclic ring the nitrogen atom may be in the form of an amide, carbamate or urea.

“Heteroaryl” means a monocyclic or bicyclic aromatic radical, where one or more, preferably one, two, or three, ring atoms are heteroatom selected from N, O, or S, the remaining ring atoms being carbon. Representative examples include, but are not limited to, pyrrolyl, thienyl (thiophenyl), thiazolyl, imidazolyl, furanyl, indolyl, isoindolyl, oxazolyl, isoxazolyl, diazolyl, pyrazolyl, triazolyl, benzothiazolyl, benzoxazolyl, quinolinyl, isoquinolinyl, pyridinyl (pyridyl), pyrimidinyl, pyrazinyl, pyridazinyl, tetrazolyl, and the like. Heteroaryl may be optionally substituted.

“Oxo” or “carbonyl” means a ═(O) group or C═O group, respectively.

The term “substituted” means that the referenced group is substituted with one or more additional group(s) individually and independently selected from groups described herein. In some embodiments, an optional substituent is selected from oxo, halogen, —CN, —NH2, —OH, —NH(CH3), —N(CH3)2, alkyl (including straight chain, branched and/or unsaturated alkyl), substituted or unsubstituted cycloalkyl, substituted or unsubstituted heterocycloalkyl, fluoroalkyl, substituted or unsubstituted heteroalkyl, substituted or unsubstituted alkoxy, fluoroalkoxy, —S-alkyl, —S(O)2-alkyl, —CONH((substituted or unsubstituted alkyl) or (substituted or unsubstituted phenyl)), —CON(H or alkyl)2, —OCON(substituted or unsubstituted alkyl)2, —NHCONH((substituted or unsubstituted alkyl) or (substituted or unsubstituted phenyl)), —NHCOalkyl, —N(substituted or unsubstituted alkyl)CO(substituted or unsubstituted alkyl), —NHCOO(substituted or unsubstituted alkyl), —C(OH)(substituted or unsubstituted alkyl)2, and —C(NH2)(substituted or unsubstituted alkyl)2. In some embodiments, by way of example, an optional substituent is selected from oxo, fluorine, chlorine, bromine, iodine, —CN, —NH2, —OH, —NH(CH3), —N(CH3)2, —CH3, —CH2CH3, —CH(CH3)2, —CF3, —CH2CF3, —OCH3, —OCH2CH3, —OCH(CH3)2, —OCF3, —OCH2CF3, —S(O)2-CH3, —CONH2, —CONHCH3, —NHCONHCH3, —COCH3, —COOH and the like. In some embodiments, substituted groups are substituted with one, two or three of the preceding groups. In some embodiments, substituted groups are substituted with one or two of the preceding groups. In some embodiments, substituted groups are substituted with one of the preceding groups. Further, unless stated to the contrary, a formula with chemical bonds shown only as solid lines and not as wedges or dashed lines contemplates each possible isomer, e.g., each enantiomer and diastereomer, and a mixture of isomers, such as racemic or scalemic mixtures.

“Addition compound” refers to a complex of two or more complete molecules in which each preserves its fundamental structure and no covalent bonds are made or broken (for example, hydrates of salts, adducts).

“Aliphatic acid” refers to acids of nonaromatic hydrocarbons. Examples of aliphatic acids include, but are not limited to, butyric acid, hexanoic acid, propionic acid, octanoic acid, and acetic acid.

“Alkene” refers to an unsaturated linear divalent hydrocarbon moiety of one to twelve, typically one to six, carbon atoms or a saturated branched divalent hydrocarbon moiety of three to twelve, typically three to six, carbon atoms. Exemplary alkene groups include, but are not limited to, methylene, ethylene, propylene, butylene, pentylene, and the like.

“Antagonist” refers to a compound or a composition that attenuates the effect of an agonist. The antagonist can directly bind reversibly or irreversibly to a region of the receptor in common with an agonist. An antagonist can also bind at a different site on the receptor or an associated ion channel. Thus, the term “antagonist” includes a functional antagonist. A “functional antagonist” refers to a compound and/or composition that reverses the effects of an agonist by means other than acting at the same receptor as the agonist, i.e., a functional antagonist causes a response in the tissue or animal which opposes the action of an agonist. Examples include agents which have opposing effects on an intracellular second messenger or on a physiologic state in an animal (for example, blood pressure).

“Biological activity” as used herein means having an effect on or eliciting or preventing a response from a living cell, tissue, organ or physiologic activity, such as, but not limited to, altering gene and/or protein expression, protein phosphorylation, cellular behavior, and/or organ function.

“Biomarker” as used herein means a measurable indicator of the severity or the presence of a particular disease state. More generally a biomarker is anything that can be used as an indicator of a particular disease state or some other physiological state of an organism.

“Carboxyl” refers to an organic functional group consisting of a carbon atom double bonded to an oxygen atom and single bonded to a hydroxyl group.

“Chiral center” (i.e., stereochemical center, stereocenter, or stereogenic center) refers to an asymmetrically substituted atom, e.g., a carbon atom to which four different groups are attached. The ultimate criterion of a chiral center, however, is nonsuperimposability of its mirror image. If an asymmetric center is present in one or more substituents, the compound may be in the form of a racemic mixture, a single enantiomer, a diastereoisomeric mixture, an enantiomeric diastereomer, a meso compound, a pure epimer, or a mixture of epimers thereof.

“Derivative” refers to a compound that is derived from some parent compound where one atom is replaced with another atom or group of atoms and usually maintains its general structure. For example, trichloromethane (chloroform) is a derivative of methane.

“Dermatitis” as used herein refers to a general or localized inflammation of the skin, either due to an inherent skin defect, direct contact with an irritating substance, virus, bacteria, animal parasite, fungus or to an allergic reaction. Symptoms of dermatitis include: redness, itching, exudations, pain, fissures, cracks, ulcers, and in some cases blistering of the skin.

“Eczema” as used herein refers to an inflammatory condition of the skin characterized by redness, itching, and oozing vesicular lesions which become scaly, crusted, or hardened.

“Epithelium (epithelia, plural) or epithelial tissues” as used herein means a type of animal tissue made up of densely packed cells that rest on a basement membrane to act as a covering of a free surface such as, but not limited to the surface of the human body; or lining of various bodily surfaces, such as but not limited to the eyes; or lining various body cavities such as but not limited the abdominal cavity; or lining the lumina of tubular structures within organs, such as but not limited to the respiratory epithelium, urogenital epithelium.

“Enantiomeric excess” refers to the difference between the amounts of enantiomers. The percentage of enantiomeric excess (% ee) can be calculated by subtracting the percentage of one enantiomer from the percentage of the other enantiomer. For example, if the % of (R)-enantiomer is 99% and % of (S)-enantiomer is 1%, the % ee of (R)-isomer is 99%-1% or 98%.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by the same or different halo atoms. The term “haloalkyl” also includes perhalogenated alkyl groups in which all alkyl hydrogen atoms are replaced by halogen atoms. Exemplary haloalkyl groups include, but are not limited to: —CH2F, —CH2Cl, —CF3, —CH2CF3, —CH2CCl3, and the like.

“Hetero-substituted alkyl” refers to an alkyl group, as defined herein, that contains one or more heteroatoms such as N, O, or S. Such heteroatoms can be hydroxy, alkoxy, amino, mono-, di-alkyl amino, thiol, alkylthiol, etc.

“Hydroxyalkyl” refers to an alkyl group, as defined herein, having one or more hydroxyl substituent(s).

As used herein, the term “inhibiting” and grammatical equivalents thereof refer to a decrease, limiting, and/or blocking of a particular action, production, function, or interaction. In one embodiment, the term refers to reducing the level of a given output or parameter to a quantity (e.g., the production of a biological active molecule) which is at least 40%, or less than the quantity in a corresponding uninhibited control. A reduced level of a given output or parameter need not, although it may, mean an absolute absence of the output or parameter. The invention does not require, and is not limited to, methods that wholly eliminate the output or parameter. Substantial inhibition can be at least 40% inhibition of the production or biological effect.

“Keto acid” refers to organic compounds that contain a carboxylic acid group and a ketone group.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N, O-dimethylhydroxylamino, and the like.

“Leukocyte or leukocytes” as used herein refer to a colorless cell that circulates in the blood and body fluids and is involved in counteracting foreign substances and infectious disease as well as being causative of inflammatory diseases. They have also been referred to as white (blood) cells. There are several types, all are amoeboid cells with a nucleus, including lymphocytes, granulocytes, mononuclear, and macrophages.

“Ligand” as used herein means a biochemical substance in the form of a nucleic acid, protein or peptide that forms a complex with another biomolecule in a cell or tissue to serve a biological purpose.

“Moderate” as used herein means to decrease or increase the quality, quantity, intensity or duration of a biological product or process.

“Parenteral” as used herein means a route of administration of a substance to an animal that occurs by other than by way of the gastrointestinal tract or alimentary canal, such as topical, intravenous (IV) injections and IV infusions.

“Peripheral blood mononuclear cell (PBMC)” as used herein means any peripheral blood cell having a round nucleus. These cells are a subset of leukocytes and consist of lymphocytes (T cells, B cells, NK cells) and mononuclear, whereas erythrocytes and platelets have no nuclei and granulocytes (neutrophils, basophils, and eosinophils) have multi-lobed nuclei.

“Pharmaceutically acceptable excipient” refers to an excipient that is useful in preparing a pharmaceutical composition that is generally safe, non-toxic, and neither biologically nor otherwise undesirable, and includes excipients that are acceptable for veterinary use as well as human pharmaceutical use.

“Pharmaceutically acceptable salt” of a compound means a salt that is pharmaceutically acceptable and that possesses the desired pharmacological activity of the parent compound. Such salts include: acid addition salts formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. A “pharmaceutically acceptable salt” of a compound also includes salts formed when an acidic proton present in the parent compound is either replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Pharmaceutically acceptable vehicle” means a carrier or inert medium used as a solvent (or diluent) in which the medicinally active agent is formulated and or administered.

The terms “pro-drug” and “prodrug” are used interchangeably herein and refer to any compound which releases an active parent drug according to Formula 1, or a pharmaceutically acceptable salt or solvate of Formula 1, in vivo when such prodrug is administered to a mammalian subject. Prodrugs of a compound of Formula 1 are prepared by modifying one or more functional group(s) present in the compound of Formula 1 in such a way that the modification(s) may be cleaved in vivo to release the parent compound. Prodrugs include compounds of Formula 1 wherein a hydroxy group in a compound of Formula 1 is bonded to any group that may be cleaved in vivo to regenerate the free hydroxyl, aliphatic alcohol, amino, or sulfhydryl group, respectively. Examples of prodrugs include, but are not limited to, esters (e.g., acetate, formate, glycol and benzoate derivatives of Formula 1) and the like. For example, the compound according to Formula 1 that is methyl 3-(4-hydroxyphenoxy)-3-methyl-butanoate can be reacted under acidic conditions with 2-hydroxybenzoic acid to produce, [4-(3-methoxy-1,-1-dimethyl-3oxo-propoxy)2-hydroxybenzoate an ester prodrug that will be hydrolyzed to 2-hydroxybenzoic acid and the starting compound by esterase enzymes in tissues. The transformation from prodrug to a compound of Formula 1, or a pharmaceutically acceptable salt or solvate thereof, may occur by various mechanisms, such as via hydrolysis in blood. A prodrug of the compound of Formula 1 may be formed in a conventional manner according to methods known in the art. A thorough discussion of prodrugs is provided by V. Stella in Pro-drugs as Novel Delivery Systems, Vol. 14 of the A.C.S. Symposium Series (Stella (1975)), and in Bioreversible Carriers in Drug Design (Roche (1987)), both of which are incorporated herein by reference.

“Pro-inflammatory cytokine” refers to a type of cytokine (i.e. a protein signaling molecule) that is secreted from leukocytes, mononuclear and other non-leukocyte cell types; such as but not limited epithelial cells, that promote inflammation by their biological effect on other cells and tissue in mammalian organisms. Non-limiting examples of pro-inflammatory cytokines are: Interleukin 1 (IL-1; IL-la & IL-1b), Interleukin 6 (IL-6), Interleukin 13 (IL-13), Tumor Necrosis Factor alpha (TNF-alpha), Interferon gamma (IFN-gamma), and Interleukin 8 (IL-8).

The term “prophylaxis” of a state, disorder, disease or condition as used herein refers to prevention of the appearance of clinical symptoms of the state, disorder, disease or condition developing in a patient that is predisposed to the state, disorder, disease, or condition.

“Protecting group” refers to a moiety, with the exception of alkyl groups, that when attached to a reactive group in a molecule masks, reduces, or prevents that reactivity. Examples of protecting groups can be found in T. W. Greene and P. G. M. Wuts, Protective Groups in Organic Synthesis, 3rd edition, John Wiley & Sons, New York, 1999, and Harrison and Harrison et al., Compendium of Synthetic Organic Methods, Vols. 1-8 (John Wiley and Sons, 1971-1996), which are incorporated by reference herein in their entirety. Representative hydroxy protecting groups include acyl groups, benzyl and trityl ethers, tetrahydropyranyl ethers, trialkylsilyl ethers, and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P, or S) to which it is attached.

As used herein the term “physiological pH” means a pH level of between 7.0-7.9. The pH is defined as the negative log₁₀ of the hydrogen ion concentration expressed in mol/L. A negative logarithmic scale is used because the numbers are all less than 1, and vary over a wide range. The pH value is most accurately determined by measuring potential difference between electrodes placed in examined and reference solutions of known pH or between measurement (glass) electrode and reference (calomel or silver chloride) electrode also known as a pH meter.

“Signal transduction” or “signaling pathway activity” refers to a biochemical causal relationship generally initiated by a protein-protein interaction such as binding of a biological active factor to a receptor, resulting in transmission of a signal from one portion of a cell to another portion of a cell. In general, the transmission can involve specific phosphorylation of one or more tyrosine, serine, or threonine residues on one or more protein components such as enzymes or transcription factors (i.e. intracellular secondary messengers) in the series of reactions causing signal transduction (often referred to as a cascade) that results in measurable changes to the cell. Penultimate cellular processes typically include nuclear events, resulting in a change in gene expression. Terminal events of signal transduction cascade result in changes in cellular activity such as but not limited to, alterations in protein products produced and/or secreted by the cell, changes in cellular behavior characteristics of division, motility, adherence, etc.

“Stereoisomer” means molecules that have the same molecular formula, molecular weight and sequence of bonded atoms (constitution), but differ in the three-dimensional orientations of their atoms in space. By definition, molecules that are stereoisomers of each other represent the same structural isomer. The chemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

“A therapeutically effective amount” means the amount of a compound that, when administered to an individual for treating a disease, is sufficient to effect such treatment for the disease, as defined below. The “therapeutically effective amount” will vary depending on the compound, the disease and its severity or affected organ or tissue and the age, weight, etc., of the individual to be treated.

“Tautomer” or “tautomeric form” means structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons. The compounds of the present invention according to Formula 1 can exist in different tautomeric states depending on the environment of the particular compound, such as the acidity or alkalinity (i.e. pH) of the solution in which they are dissolved.

“Topical or topically applied” as used herein means the direct delivery or application of the active drug ingredient such as compounds according to Formula 1 directly to the apical surface of a cell or to the exposed surface of a tissue or organ. As used herein most often topical administration means application to epithelial surfaces such as the skin, eyes or mucous membranes to treat ailments by means of a spray, cream, ointment, shampoo, lotion, solution or other suitable delivery solvent or vehicle at physiological pH. Various methods of topical drug delivery such as; but not limited to mechanical application, instillation, inhalation, patches, and the like are known to those skilled in the art, are commonly used for topical application, and are implicit in the concept of topical application as used herein.

“Treating” or “treatment” of a disease as used herein means inhibiting the disease, i.e. arresting or reducing the pathophysiologic process or processes of the disease or its clinical symptoms; or relieving the disease, i.e., causing regression of the pathophysiologic process or processes of disease or reducing the clinical manifestations of the pathophysiologic process or processes of the specific disease.

In some embodiments, a compound of the disclosure is present in a composition as a salt. In some embodiments, salts are obtained by reacting a compound of the disclosure with acids. In some other embodiments, pharmaceutically acceptable salts are obtained by reacting a compound of the disclosure with a base. In other embodiments, the compounds are used as free-acid or free-base form in the manufacture of the compositions described herein. The type of salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, the lipid modulating compound described herein are reacted with an organic base, such as, but not limited to, ethanolamine, diethanolamine, triethanolamine, methylamine, dimethylamine, trimethylamine, ethylamine, diethylamine, triethylamine, N-methylglucamine, dicyclohexylamine, tris(hydroxymethyl)methylamine. In other cases, the compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

In the scope of the embodiments, the compounds described herein include further forms of the compounds such as pharmaceutically acceptable salts, solvates (including hydrates), amorphous phases, partially crystalline and crystalline forms (including all polymorphs), prodrugs, metabolites, N-oxides, isotopically-labeled, epimers, pure epimers, epimer mixtures, enantiomers including but not limited to single enantiomers and enantiomeric diastereomers, meso compounds, stereoisomers, racemic mixtures and diastereoisomeric mixtures. Compounds described herein having one or more double bonds include cis/trans isomers, E/Z isomers and geometric isomers.

In some embodiments, sites on the compounds disclosed herein are susceptible to various metabolic reactions. Therefore incorporation of appropriate substituents at the places of metabolic reactions will reduce, minimize or eliminate the metabolic pathways. In specific embodiments, the appropriate substituent to decrease or eliminate the susceptibility of the aromatic ring to metabolic reactions is, by way of example only, a halogen, deuterium or an alkyl group. Examples of such substituents can be found in Burger's Medicinal Chemistry, Drug Discovery and Development, 8 Volume Set (Abraham (2010)) and in Foye's Principles of Medicinal Chemistry (Lemke (2012)).

In some embodiments, sites on the compounds disclosed herein are not susceptible to various metabolic reactions. Therefore incorporation of appropriate substituents at or near or distant from the places of a lack of metabolic reactions will modulate, enhance, or maximize the metabolic pathways. In specific embodiments, the appropriate substituent (metabolic handle) to enhance, or maximize the susceptibility of the aromatic ring to metabolic reactions is, by way of example only, is a phenolic or methoxy or carboxylate group. Examples of such substituents can be found in Burger's Medicinal Chemistry, Drug Discovery and Development, 8 Volume Set (Abraham (2010)) and in Foye's Principles of Medicinal Chemistry (Lemke (2012)).

Throughout the specification, groups and substituents thereof can be chosen by one skilled in the field to provide stable moieties and compounds.

### Synthesis of the Compounds

In general, compounds of Formula I may be prepared using a number of methods known in the chemical arts, particularly in light of the description contained herein, in combination with the knowledge of the skilled artisan. Various starting materials, intermediates, and reagents may be purchased from commercial sources or made according to literature methods or adaptations thereof. Although other reagents, compounds or methods can be used in practice or testing, generalized methods for the preparation of the compound of Formula I are illustrated by the following descriptions and reaction Schemes. The methods disclosed herein, including those outlined in the Schemes, descriptions, and Examples are for intended for illustrative purposes and are not to be construed in any manner as limitations thereon. Various changes and modifications will be obvious to those of skill in the art given the benefit of the present disclosure and are deemed to be within the spirit and scope of the present disclosure as further defined in the appended claims.

Although specific embodiments of various aspects of the invention will be described with reference to the Schemes, Preparations and/or Examples, it should be understood that such embodiments are by way of example only and are merely illustrative of a small number of the many possible specific embodiments which can represent applications of the principles of the present disclosure. The starting materials used for the synthesis of compounds described herein can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March's Advanced Organic Chemistry: Reactions, Mechanisms, and Structure (Smith (2013)), Design and Strategy in Organic Synthesis (Hanessian (2013)) Greene's Protective Groups in Organic Synthesis (Wuts (2006)) and Fiesers' Reagents for Organic Synthesis (Volumes 1-27) (Ho (2013)), each of which are incorporated by reference in their entirety.

General methods for the preparation of the compounds as disclosed herein may be derived from known reactions in the field, and the reactions may be modified by the use of appropriate reagents and conditions, as would be recognized by the skilled person, for the introduction of the various moieties found in the formulae as provided herein.

The intermediate products described can be recovered by extraction, evaporation, or other techniques known in the art. The crude materials may then be optionally purified by chromatography, HPLC, recrystallization, trituration, distillation, or other techniques known in the art. In the discussions below, the following abbreviations were used: EtOH (ethanol) NaOH (sodium ethoxide), DMSO (dimethylsulfoxide), MOM (methoxymethyl), THF (tetrahydrofuran), Dess-Martin (Dess-Martin Periodinane) and TBS (tert-butyldimethylsilyl).

As would be appreciated by those skilled in the art, some of the methods useful for the preparation of such compounds, as discussed above, may require protection of a particular functionality, e.g., to prevent interference by such functionality in reactions at other sites within the molecule or to preserve the integrity of such functionality. The need for, and type of, such protection is readily determined by one skilled in the art, and will vary depending on, for example, the nature of the functionality and the conditions of the selected preparation method. Methods of introducing and removing protecting groups are well known to those of ordinary skill in the art and are described in Greene's Protective Groups in Organic Synthesis (Wuts (2006)). Alternate reagents, starting materials, as well as methods for optimizing or adapting the procedures described herein would also be readily determined by one skilled in the art.

### The C═O Analogs

The traditional method of benzimidazole synthesis based on the condensation of a suitably substituted alpha carbonyl acid chloride with a suitably substituted aromatic 1,2 diamine to provide the corresponding benzimidazole can be used to synthesize compounds of this class, such as those in Table 1, as shown in Scheme 1 below.

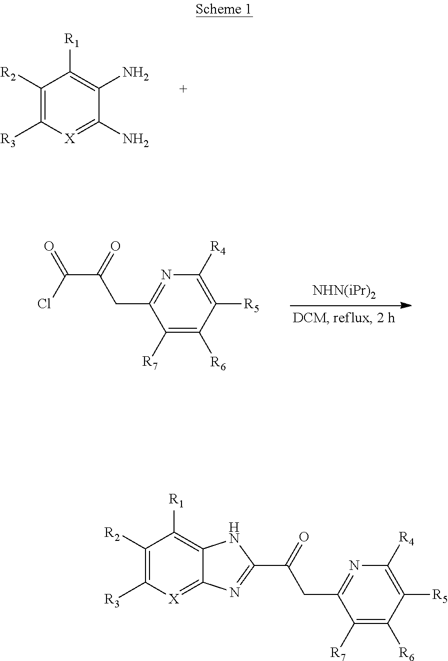

Similar pathways are available using appropriate activated esters instead of the acid chloride as well as a version with a protected alpha carbonyl group.

An umpolung methodology can also provide this class of analogs directly from a chlorinated benzimidazole as shown in Scheme 2. Such substituted chloroimidazoles are widely available commercially.

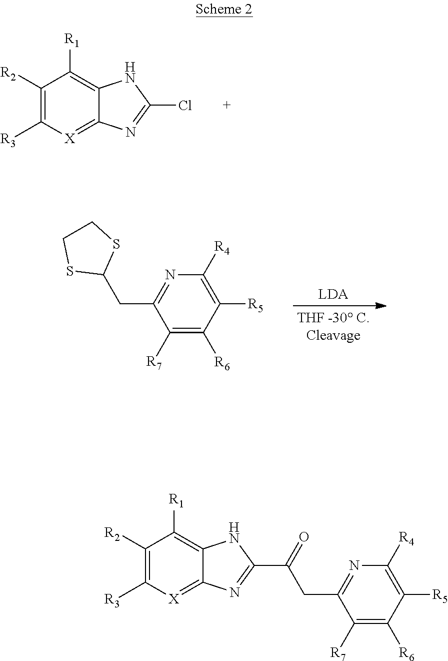

### The CHOH Analogs

Sodium Borohydride reduction of the C═O analogs prepared via Schemes 1 or 2 leads directly to the CHOH analogs, including but not limited to those listed in Table 2, as shown in Scheme 3. Other hydride reducing agents are envisaged such as one of the Selectides (L N or K Selectide) or LAH. Asymmetric reduction with a suitable optically active borane or aluminium hydride reagent can give the individual enantiomers.

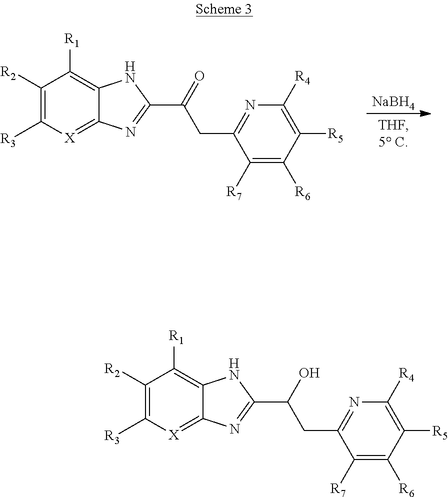

### The C(alkyl)OH Analogs

The traditional method of carbonyl alkylation with the action Methyl Lithium or Methyl Magnesium Bromide on the C═O analogs available from Schemes 1 or 2 can be used to synthesize compounds of this class, including but not limited to those shown in Table 3, as shown in Scheme 4 below.

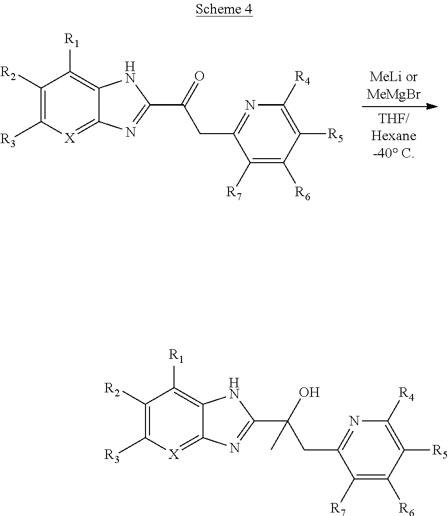

Enantiomers are available from chiral HPLC methods.

### The S═O Analogs

The methodology used to synthesize esomeprazole can be used to synthesis a wide range of S═O analogs, including but not limited to those shown in Table 4, as indicated below in Scheme 5 (Kohl et al (1992)).

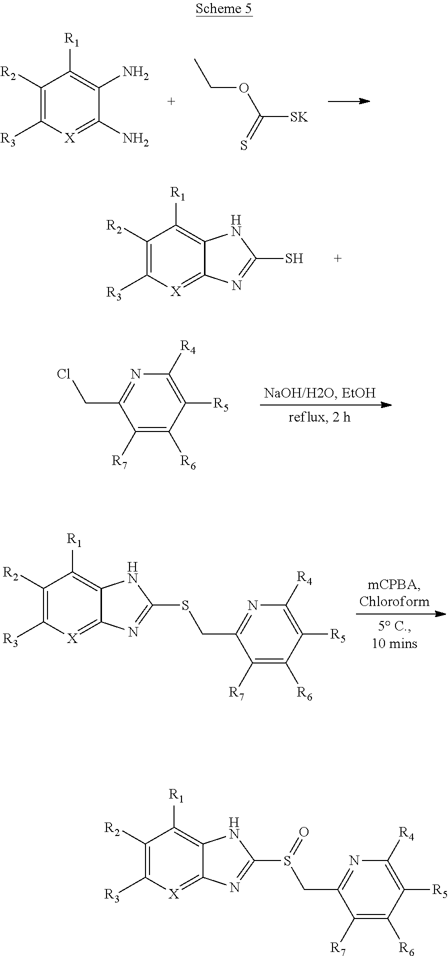

Rather than going through a lengthy separation protocol involving diastereomeric derivatives of these sulfoxides an asymmetric synthesis of the enantiomeric analogs starting from the S precursor is more efficient, including but not limited to those shown in Table 5, as indicated below in Scheme 6 (Lindberg et al (1998), Cotton et al (1998)).

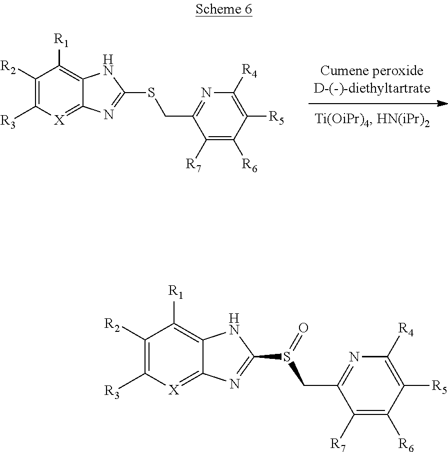

Reversing the configuration from D-diethyl tartrate to L-diethyl tartrate reverses the absolute configuration of the products, including but not limited to those shown in Table 6, as indicated below in Scheme 7 (Lindberg et al (1998), Cotton et al (1998)).

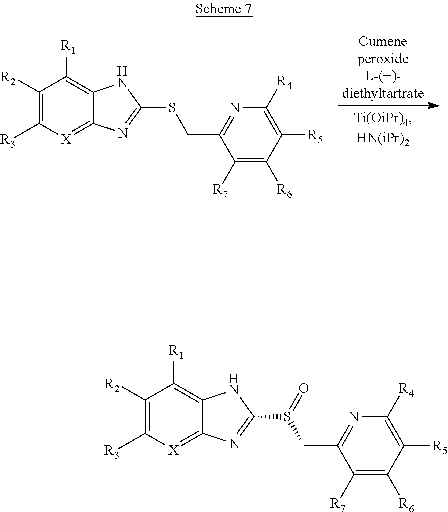

### The SO2 Analogs

Full oxidation of the S analogs gives the sulfones, including but not limited to those shown in Table 7, as indicated below in Scheme 8. Other oxidation conditions such as sodium periodate are envisaged.

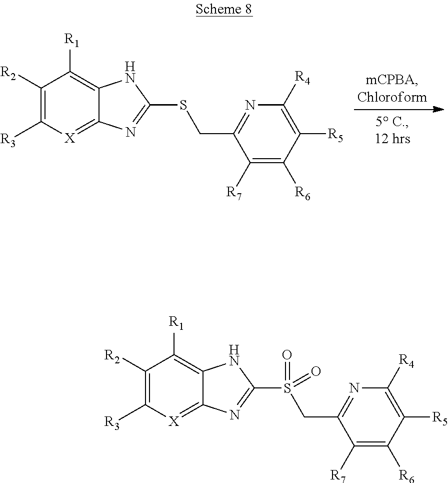

The modular syntheses of Schemes 1 through 8 can all be adapted to automated synthesis platforms, focused library platforms, solid phase organic synthesis platforms, combinatorial chemistry platforms, microwave chemistry platforms, DNA encoded library platforms and other modern variants of synthetic organic chemistry suitable for high throughput.

The following Tables 1 through 7 illustrate the analogs prepared.

TABLE 1
Structure

| | |
|---|---|
| 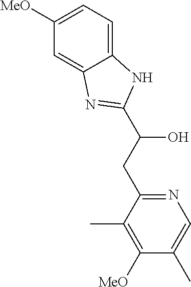 [SMILES: COc1ccc2[nH]c(C(O)Cc3ncc(C)c(OC)c3C)nc2c1] | 1) |
| 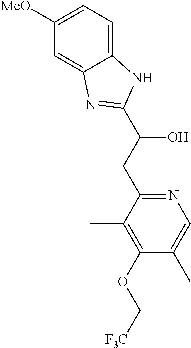 [SMILES: COc1ccc2[nH]c(C(O)Cc3ncc(C)c(OCC(F)(F)F)c3C)nc2c1] | 2) |
| 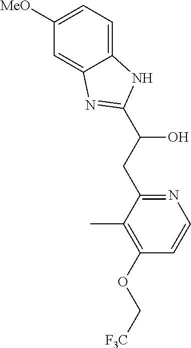 [SMILES: COc1ccc2[nH]c(C(O)Cc3nccc(OCC(F)(F)F)c3C)nc2c1] | 3) |
| 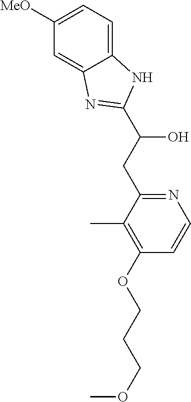 [SMILES: COCCCOc1ccnc(CC(O)c2nc3cc(OC)ccc3[nH]2)c1C] | 4) |
| 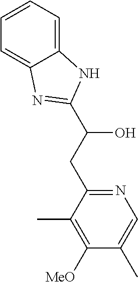 [SMILES: COc1c(C)cnc(CC(O)c2nc3ccccc3[nH]2)c1C] | 5) |
| 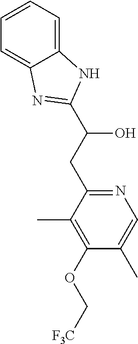 [SMILES: Cc1cnc(CC(O)c2nc3ccccc3[nH]2)c(C)c1OCC(F)(F)F] | 6) |
| 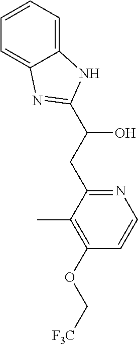 [SMILES: Cc1c(OCC(F)(F)F)ccnc1CC(O)c1nc2ccccc2[nH]1] | 7) |
| 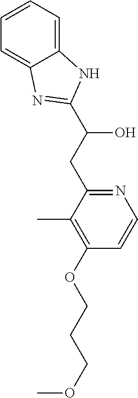 [SMILES: COCCCOc1ccnc(CC(O)c2nc3ccccc3[nH]2)c1C] | 8) |
| 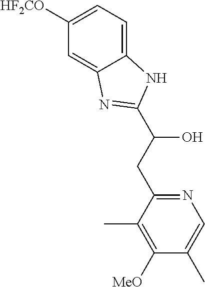 | 9) |
| 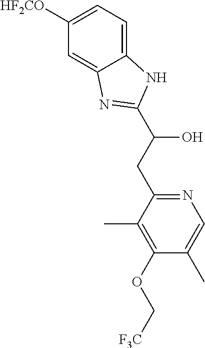 | 10) |
| 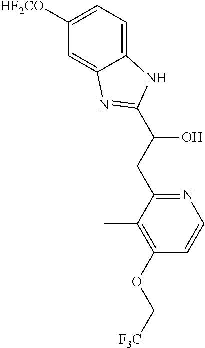 | 11) |
| 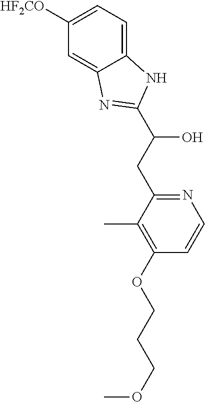 | 12) |
| 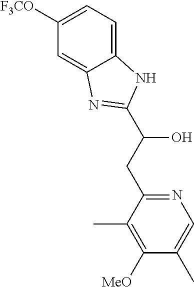 [SMILES: COc1c(C)cnc(CC(O)c2nc3cc(OC(F)(F)F)ccc3[nH]2)c1C] | 13) |
| 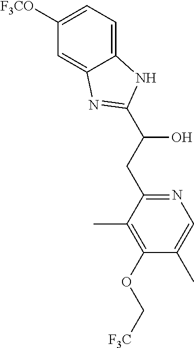 [SMILES: Cc1cnc(CC(O)c2nc3cc(OC(F)(F)F)ccc3[nH]2)c(C)c1OCC(F)(F)F] | 14) |
| 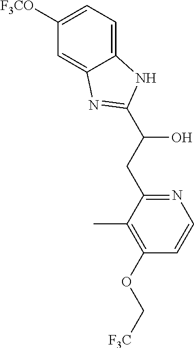 [SMILES: Cc1c(OCC(F)(F)F)ccnc1CC(O)c1nc2cc(OC(F)(F)F)ccc2[nH]1] | 15) |
| 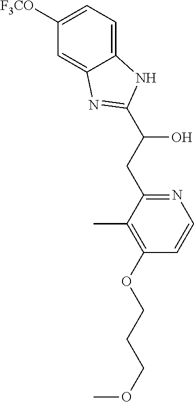 [SMILES: COCCCOc1ccnc(CC(O)c2nc3cc(OC(F)(F)F)ccc3[nH]2)c1C] | 16) |
| 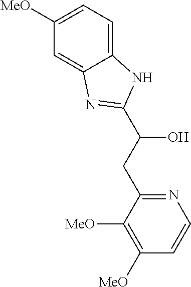 [SMILES: COc1ccc2[nH]c(C(O)Cc3nccc(OC)c3OC)nc2c1] | 17) |
| 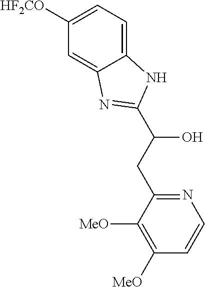 | 18) |
| 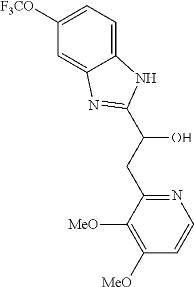 [SMILES: COc1ccnc(CC(O)c2nc3cc(OC(F)(F)F)ccc3[nH]2)c1OC] | 19) |
| 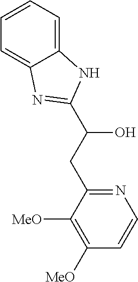 [SMILES: COc1ccnc(CC(O)c2nc3ccccc3[nH]2)c1OC] | 20) |
| 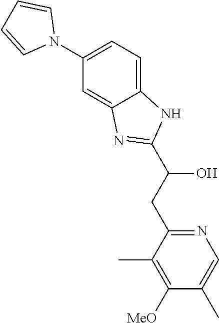 [SMILES: COc1c(C)cnc(CC(O)c2nc3cc(-n4cccc4)ccc3[nH]2)c1C] | 21) |
| 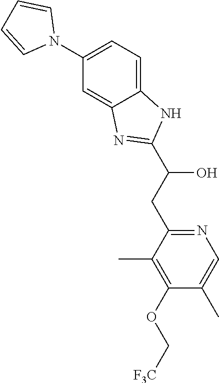 [SMILES: Cc1cnc(CC(O)c2nc3cc(-n4cccc4)ccc3[nH]2)c(C)c1OCC(F)(F)F] | 22) |
| 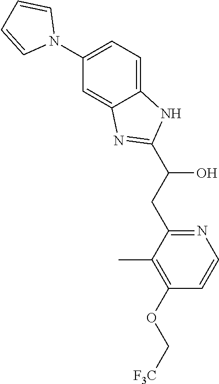 [SMILES: Cc1c(OCC(F)(F)F)ccnc1CC(O)c1nc2cc(-n3cccc3)ccc2[nH]1] | 23) |
| 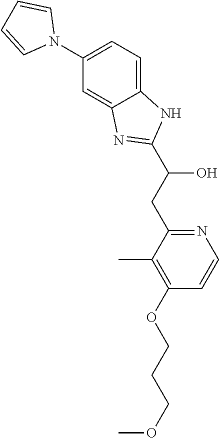 [SMILES: COCCCOc1ccnc(CC(O)c2nc3cc(-n4cccc4)ccc3[nH]2)c1C] | 24) |
| 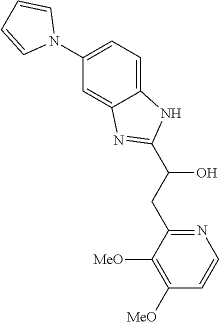 [SMILES: COc1ccnc(CC(O)c2nc3cc(-n4cccc4)ccc3[nH]2)c1OC] | 25) |
| 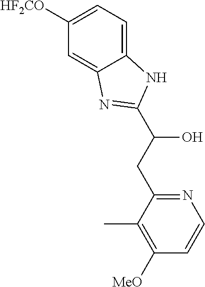 | 26) |
| 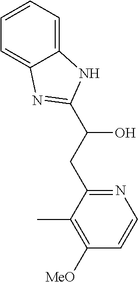 [SMILES: COc1ccnc(CC(O)c2nc3ccccc3[nH]2)c1C] | 27) |
| 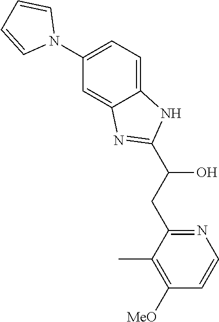 [SMILES: COc1ccnc(CC(O)c2nc3cc(-n4cccc4)ccc3[nH]2)c1C] | 28) |
| 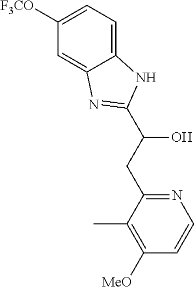 [SMILES: COc1ccnc(CC(O)c2nc3cc(OC(F)(F)F)ccc3[nH]2)c1C] | 29) |
| 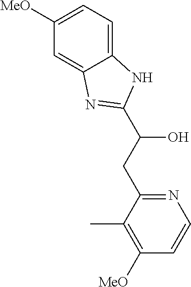 [SMILES: COc1ccc2[nH]c(C(O)Cc3nccc(OC)c3C)nc2c1] | 30) |

TABLE 2
Structure

| | |
|---|---|
| 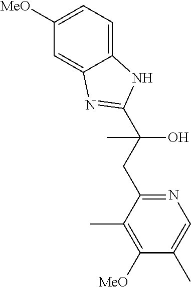 [SMILES: COc1ccc2[nH]c(C(C)(O)Cc3ncc(C)c(OC)c3C)nc2c1] | 31) |
| 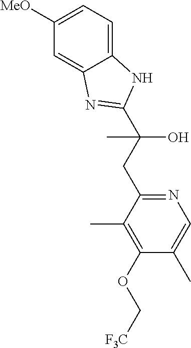 [SMILES: COc1ccc2[nH]c(C(C)(O)Cc3ncc(C)c(OCC(F)(F)F)c3C)nc2c1] | 32) |
| 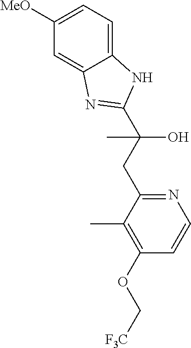 [SMILES: COc1ccc2[nH]c(C(C)(O)Cc3nccc(OCC(F)(F)F)c3C)nc2c1] | 33) |
| 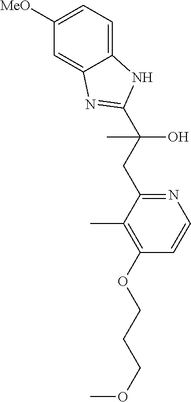 [SMILES: COCCCOc1ccnc(CC(C)(O)c2nc3cc(OC)ccc3[nH]2)c1C] | 34) |
| 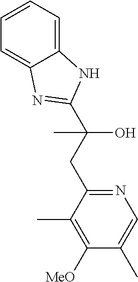 [SMILES: COc1c(C)cnc(CC(C)(O)c2nc3ccccc3[nH]2)c1C] | 35) |
| 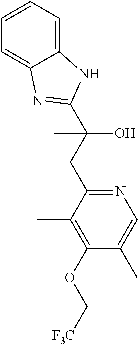 [SMILES: Cc1cnc(CC(C)(O)c2nc3ccccc3[nH]2)c(C)c1OCC(F)(F)F] | 36) |
| 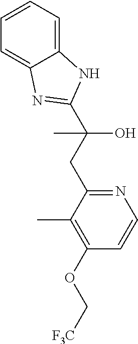 [SMILES: Cc1c(OCC(F)(F)F)ccnc1CC(C)(O)c1nc2ccccc2[nH]1] | 37) |
| 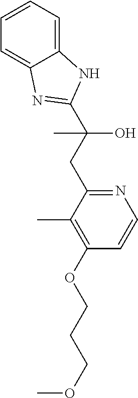 [SMILES: COCCCOc1ccnc(CC(C)(O)c2nc3ccccc3[nH]2)c1C] | 38) |
| 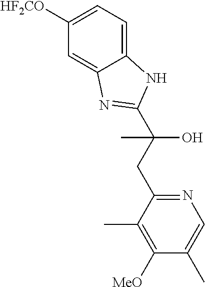 | 39) |
| 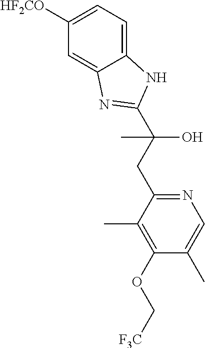 | 40) |
| 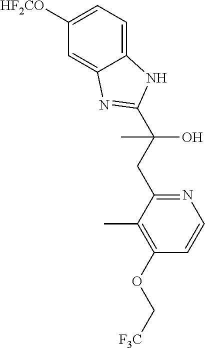 | 41) |
| 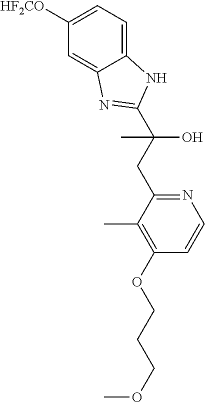 | 42) |
| 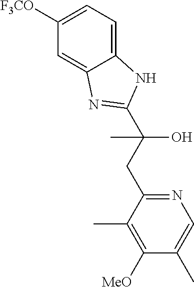 [SMILES: COc1c(C)cnc(CC(C)(O)c2nc3cc(OC(F)(F)F)ccc3[nH]2)c1C] | 43) |
| 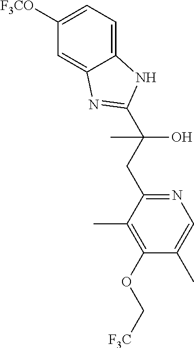 [SMILES: Cc1cnc(CC(C)(O)c2nc3cc(OC(F)(F)F)ccc3[nH]2)c(C)c1OCC(F)(F)F] | 44) |
| 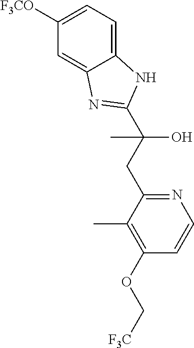 [SMILES: Cc1c(OCC(F)(F)F)ccnc1CC(C)(O)c1nc2cc(OC(F)(F)F)ccc2[nH]1] | 45) |
| 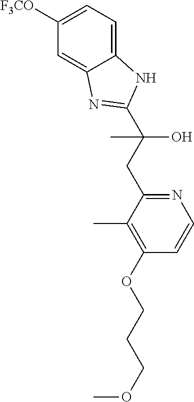 [SMILES: COCCCOc1ccnc(CC(C)(O)c2nc3cc(OC(F)(F)F)ccc3[nH]2)c1C] | 46) |
| 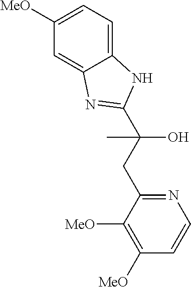 [SMILES: COc1ccc2[nH]c(C(C)(O)Cc3nccc(OC)c3OC)nc2c1] | 47) |
| 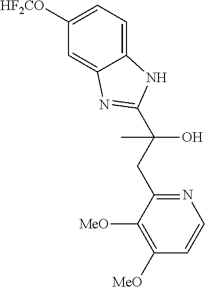 | 48) |
| 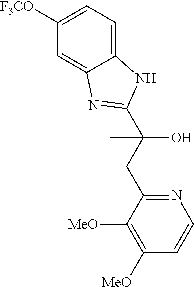 [SMILES: COc1ccnc(CC(C)(O)c2nc3cc(OC(F)(F)F)ccc3[nH]2)c1OC] | 49) |
| 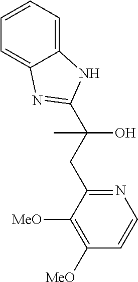 [SMILES: COc1ccnc(CC(C)(O)c2nc3ccccc3[nH]2)c1OC] | 50) |
| 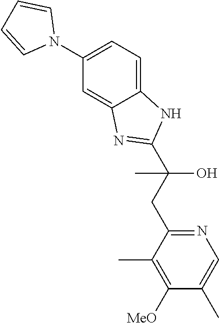 [SMILES: COc1c(C)cnc(CC(C)(O)c2nc3cc(-n4cccc4)ccc3[nH]2)c1C] | 51) |
| 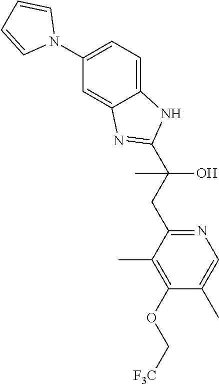 [SMILES: Cc1cnc(CC(C)(O)c2nc3cc(-n4cccc4)ccc3[nH]2)c(C)c1OCC(F)(F)F] | 52) |
| 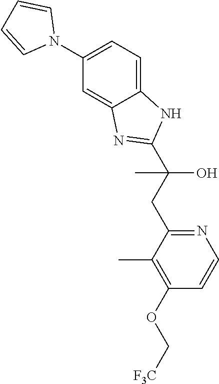 [SMILES: Cc1c(OCC(F)(F)F)ccnc1CC(C)(O)c1nc2cc(-n3cccc3)ccc2[nH]1] | 53) |
| 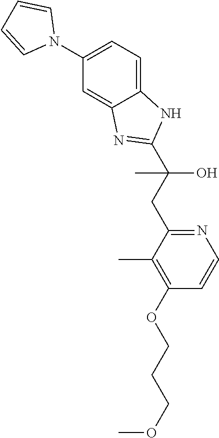 [SMILES: COCCCOc1ccnc(CC(C)(O)c2nc3cc(-n4cccc4)ccc3[nH]2)c1C] | 54) |
| 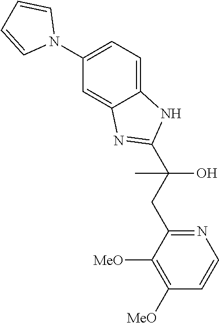 [SMILES: COc1ccnc(CC(C)(O)c2nc3cc(-n4cccc4)ccc3[nH]2)c1OC] | 55) |
| 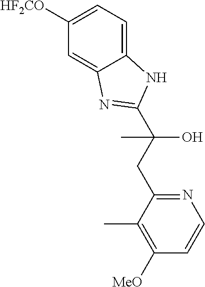 | 56) |
| 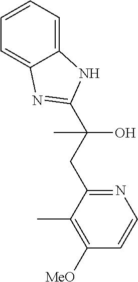 [SMILES: COc1ccnc(CC(C)(O)c2nc3ccccc3[nH]2)c1C] | 57) |
| 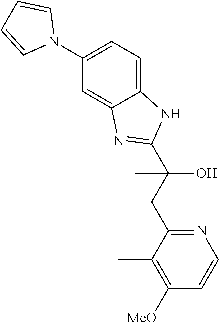 [SMILES: COc1ccnc(CC(C)(O)c2nc3cc(-n4cccc4)ccc3[nH]2)c1C] | 58) |
| 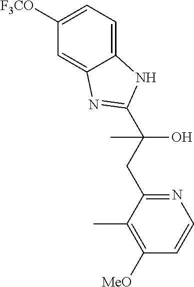 [SMILES: COc1ccnc(CC(C)(O)c2nc3cc(OC(F)(F)F)ccc3[nH]2)c1C] | 59) |
| 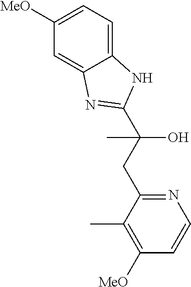 [SMILES: COc1ccc2[nH]c(C(C)(O)Cc3nccc(OC)c3C)nc2c1] | 60) |

TABLE 3
Structure

| | |
|---|---|
| 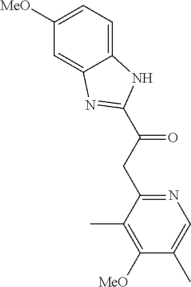 [SMILES: COc1ccc2[nH]c(C(=O)Cc3ncc(C)c(OC)c3C)nc2c1] | 61) |
| 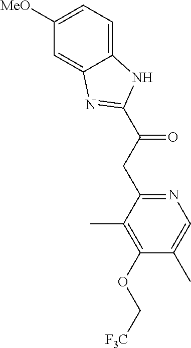 [SMILES: COc1ccc2[nH]c(C(=O)Cc3ncc(C)c(OCC(F)(F)F)c3C)nc2c1] | 62) |
| 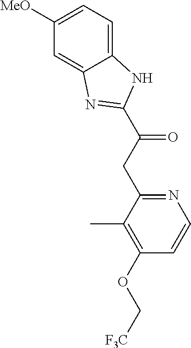 [SMILES: COc1ccc2[nH]c(C(=O)Cc3nccc(OCC(F)(F)F)c3C)nc2c1] | 63) |
| 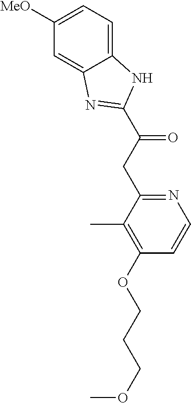 [SMILES: COCCCOc1ccnc(CC(=O)c2nc3cc(OC)ccc3[nH]2)c1C] | 64) |
| 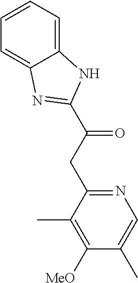 [SMILES: COc1c(C)cnc(CC(=O)c2nc3ccccc3[nH]2)c1C] | 65) |
| 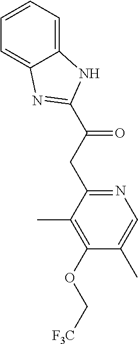 [SMILES: Cc1cnc(CC(=O)c2nc3ccccc3[nH]2)c(C)c1OCC(F)(F)F] | 66) |
| 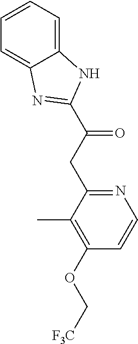 [SMILES: Cc1c(OCC(F)(F)F)ccnc1CC(=O)c1nc2ccccc2[nH]1] | 67) |
| 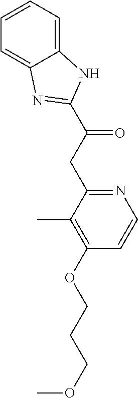 [SMILES: COCCCOc1ccnc(CC(=O)c2nc3ccccc3[nH]2)c1C] | 68) |
| 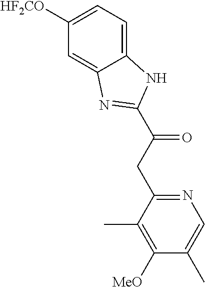 | 69) |
| 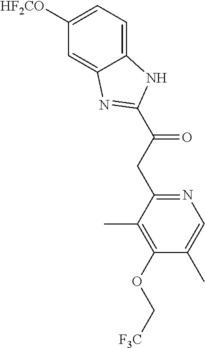 | 70) |
| 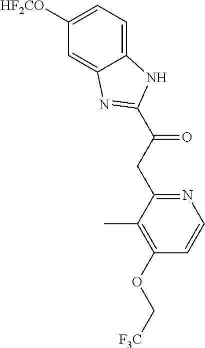 | 71) |
| 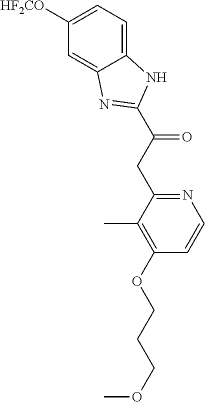 | 72) |
| 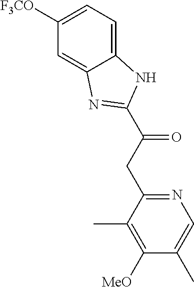 [SMILES: COc1c(C)cnc(CC(=O)c2nc3cc(OC(F)(F)F)ccc3[nH]2)c1C] | 73) |
| 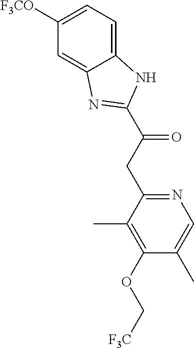 [SMILES: Cc1cnc(CC(=O)c2nc3cc(OC(F)(F)F)ccc3[nH]2)c(C)c1OCC(F)(F)F] | 74) |
| 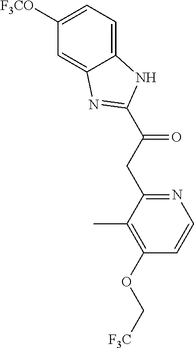 [SMILES: Cc1c(OCC(F)(F)F)ccnc1CC(=O)c1nc2cc(OC(F)(F)F)ccc2[nH]1] | 75) |
| 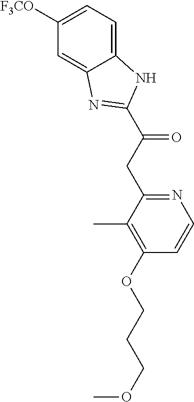 [SMILES: COCCCOc1ccnc(CC(=O)c2nc3cc(OC(F)(F)F)ccc3[nH]2)c1C] | 76) |
| 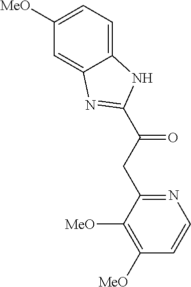 [SMILES: COc1ccc2[nH]c(C(=O)Cc3nccc(OC)c3OC)nc2c1] | 77) |
| 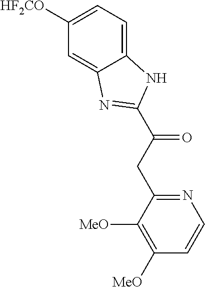 | 78) |
| 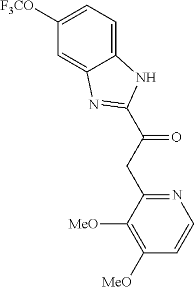 [SMILES: COc1ccnc(CC(=O)c2nc3cc(OC(F)(F)F)ccc3[nH]2)c1OC] | 79) |
| 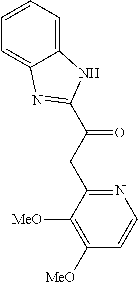 [SMILES: COc1ccnc(CC(=O)c2nc3ccccc3[nH]2)c1OC] | 80) |
| 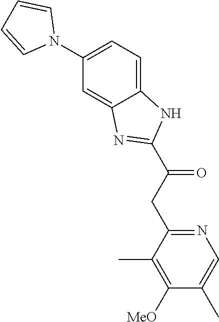 [SMILES: COc1c(C)cnc(CC(=O)c2nc3cc(-n4cccc4)ccc3[nH]2)c1C] | 81) |
| 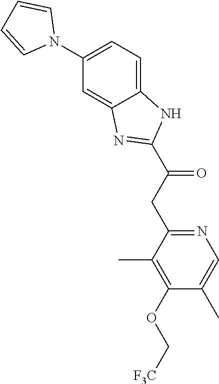 [SMILES: Cc1cnc(CC(=O)c2nc3cc(-n4cccc4)ccc3[nH]2)c(C)c1OCC(F)(F)F] | 82) |
| 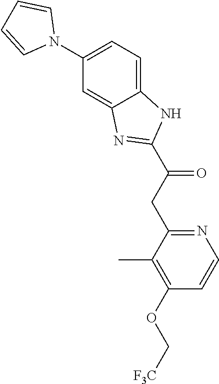 [SMILES: Cc1c(OCC(F)(F)F)ccnc1CC(=O)c1nc2cc(-n3cccc3)ccc2[nH]1] | 83) |
| 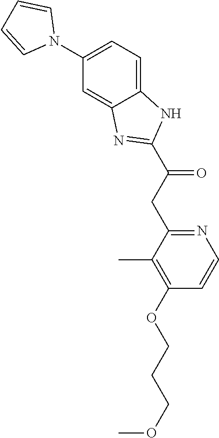 [SMILES: COCCCOc1ccnc(CC(=O)c2nc3cc(-n4cccc4)ccc3[nH]2)c1C] | 84) |
| 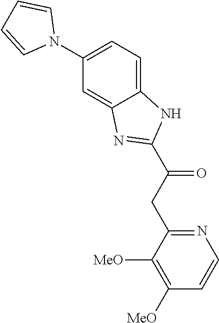 [SMILES: COc1ccnc(CC(=O)c2nc3cc(-n4cccc4)ccc3[nH]2)c1OC] | 85) |
| 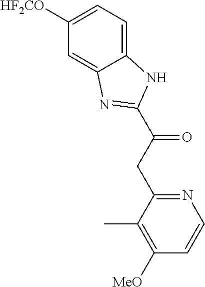 | 86) |
| 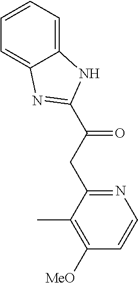 [SMILES: COc1ccnc(CC(=O)c2nc3ccccc3[nH]2)c1C] | 87) |
| 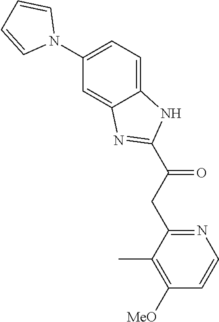 [SMILES: COc1ccnc(CC(=O)c2nc3cc(-n4cccc4)ccc3[nH]2)c1C] | 88) |
| 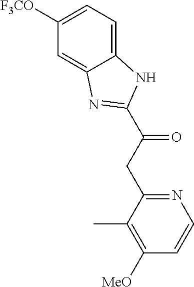 [SMILES: COc1ccnc(CC(=O)c2nc3cc(OC(F)(F)F)ccc3[nH]2)c1C] | 89) |
| 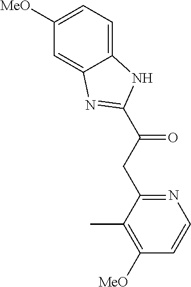 [SMILES: COc1ccc2[nH]c(C(=O)Cc3nccc(OC)c3C)nc2c1] | 90) |

TABLE 4
Structure

| | |
|---|---|
| 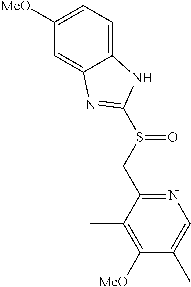 [SMILES: COc1ccc2[nH]c(S(=O)Cc3ncc(C)c(OC)c3C)nc2c1] | 91) |
| 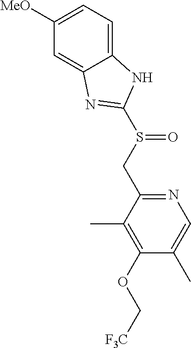 [SMILES: COc1ccc2[nH]c(S(=O)Cc3ncc(C)c(OCC(F)(F)F)c3C)nc2c1] | 92) |
| 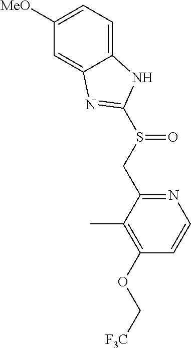 [SMILES: COc1ccc2[nH]c(S(=O)Cc3nccc(OCC(F)(F)F)c3C)nc2c1] | 93) |
| 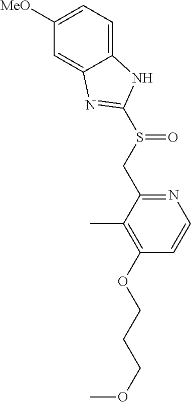 [SMILES: COCCCOc1ccnc(CS(=O)c2nc3cc(OC)ccc3[nH]2)c1C] | 94) |
| 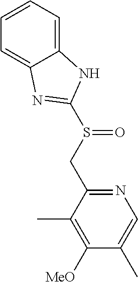 [SMILES: COc1c(C)cnc(CS(=O)c2nc3ccccc3[nH]2)c1C] | 95) |
| 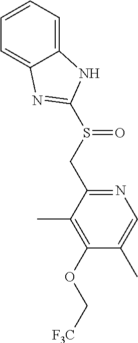 [SMILES: Cc1cnc(CS(=O)c2nc3ccccc3[nH]2)c(C)c1OCC(F)(F)F] | 96) |
| 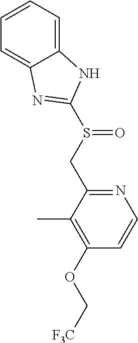 [SMILES: Cc1c(OCC(F)(F)F)ccnc1CS(=O)c1nc2ccccc2[nH]1] | 97) |
| 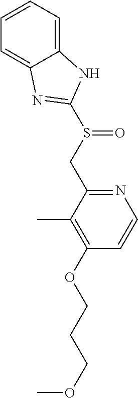 [SMILES: COCCCOc1ccnc(CS(=O)c2nc3ccccc3[nH]2)c1C] | 98) |
| 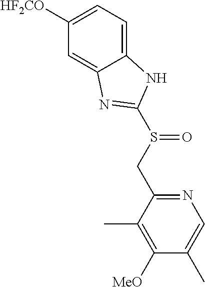 | 99) |
| 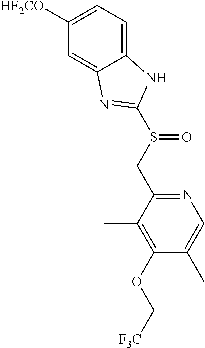 | 100) |
| 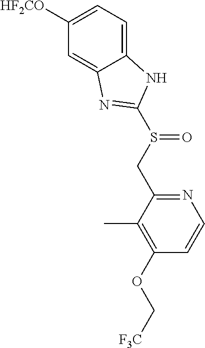 | 101) |
| 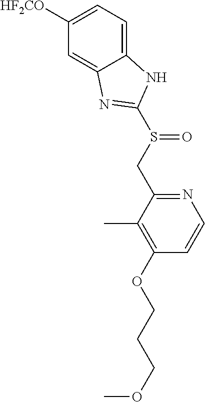 | 102) |
| 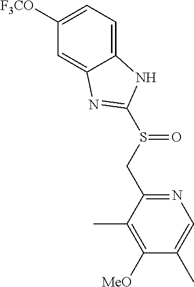 [SMILES: COc1c(C)cnc(CS(=O)c2nc3cc(OC(F)(F)F)ccc3[nH]2)c1C] | 103) |
| 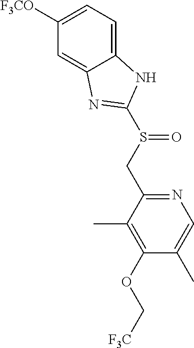 [SMILES: Cc1cnc(CS(=O)c2nc3cc(OC(F)(F)F)ccc3[nH]2)c(C)c1OCC(F)(F)F] | 104) |
| 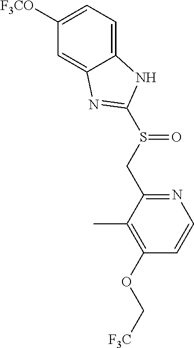 [SMILES: Cc1c(OCC(F)(F)F)ccnc1CS(=O)c1nc2cc(OC(F)(F)F)ccc2[nH]1] | 105) |
| 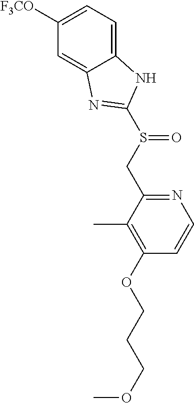 [SMILES: COCCCOc1ccnc(CS(=O)c2nc3cc(OC(F)(F)F)ccc3[nH]2)c1C] | 106) |
| 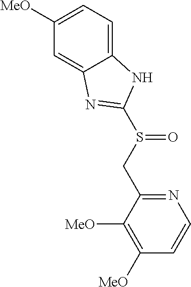 [SMILES: COc1ccc2[nH]c(S(=O)Cc3nccc(OC)c3OC)nc2c1] | 107) |
| 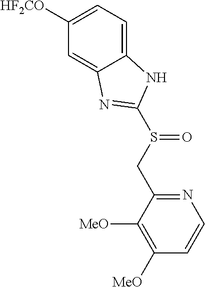 | 108) |
| 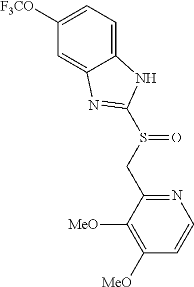 [SMILES: COc1ccnc(CS(=O)c2nc3cc(OC(F)(F)F)ccc3[nH]2)c1OC] | 109) |
| 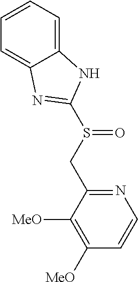 [SMILES: COc1ccnc(CS(=O)c2nc3ccccc3[nH]2)c1OC] | 110) |
| 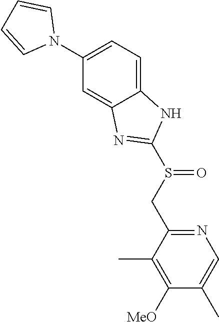 [SMILES: COc1c(C)cnc(CS(=O)c2nc3cc(-n4cccc4)ccc3[nH]2)c1C] | 111) |
| 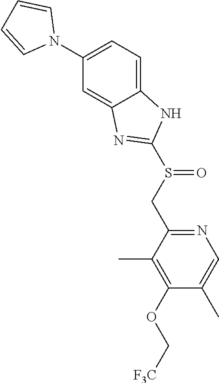 [SMILES: Cc1cnc(CS(=O)c2nc3cc(-n4cccc4)ccc3[nH]2)c(C)c1OCC(F)(F)F] | 112) |
| 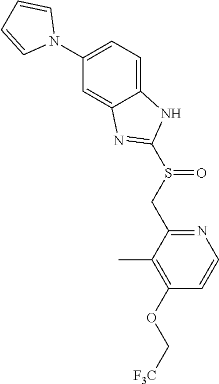 [SMILES: Cc1c(OCC(F)(F)F)ccnc1CS(=O)c1nc2cc(-n3cccc3)ccc2[nH]1] | 113) |
| 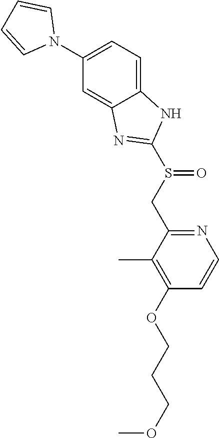 [SMILES: COCCCOc1ccnc(CS(=O)c2nc3cc(-n4cccc4)ccc3[nH]2)c1C] | 114) |
| 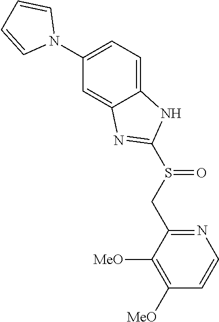 [SMILES: COc1ccnc(CS(=O)c2nc3cc(-n4cccc4)ccc3[nH]2)c1OC] | 115) |
| 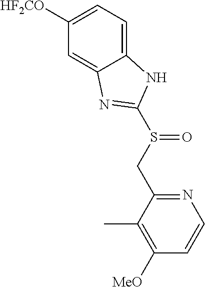 | 116) |
| 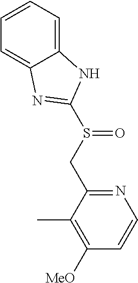 [SMILES: COc1ccnc(CS(=O)c2nc3ccccc3[nH]2)c1C] | 117) |
| 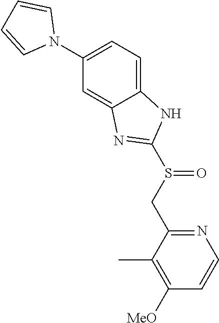 [SMILES: COc1ccnc(CS(=O)c2nc3cc(-n4cccc4)ccc3[nH]2)c1C] | 118) |
| 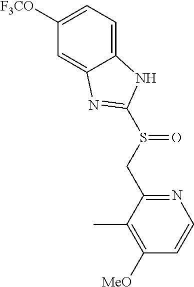 [SMILES: COc1ccnc(CS(=O)c2nc3cc(OC(F)(F)F)ccc3[nH]2)c1C] | 119) |
| 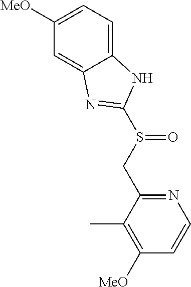 [SMILES: COc1ccc2[nH]c(S(=O)Cc3nccc(OC)c3C)nc2c1] | 120) |

TABLE 5
Structure

| | |
|---|---|
| 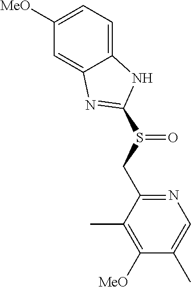 | 121) |
| 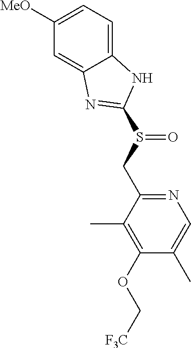 | 122) |
| 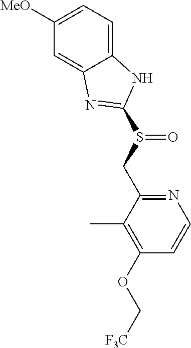 | 123) |
| 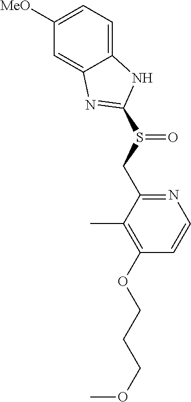 | 124) |
| 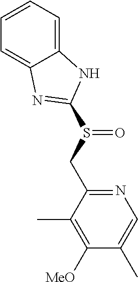 | 125) |
| 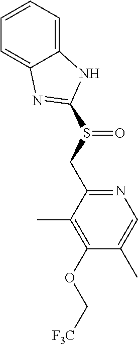 | 126) |
| 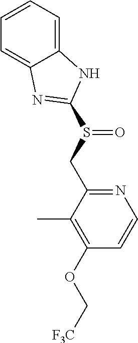 | 127) |
| 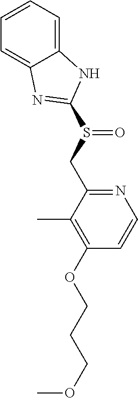 | 128) |
| 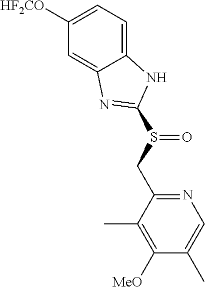 | 129) |
| 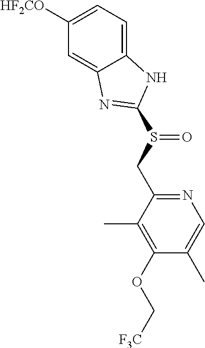 | 130) |
| 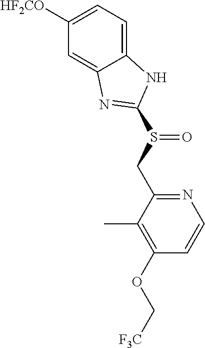 | 131) |
| 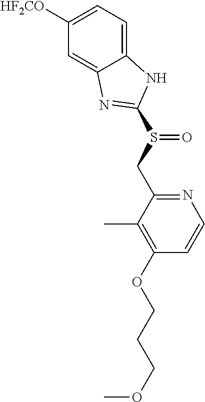 | 132) |
| 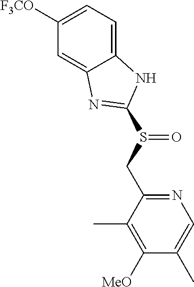 | 133) |
| 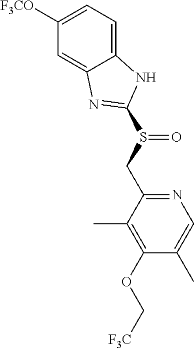 | 134) |
| 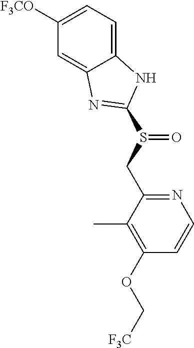 | 135) |
| 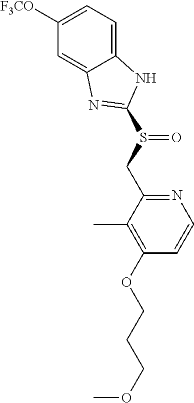 | 136) |
| 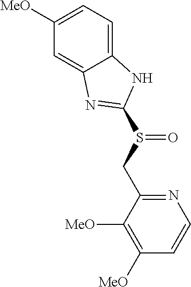 | 137) |
| 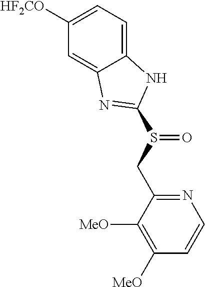 | 138) |
| 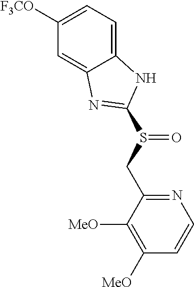 | 139) |
| 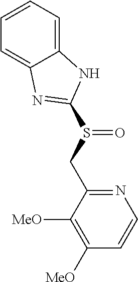 | 140) |
| 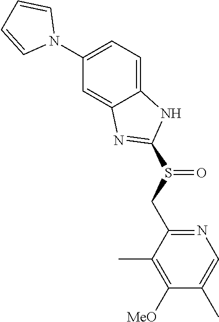 | 141) |
| 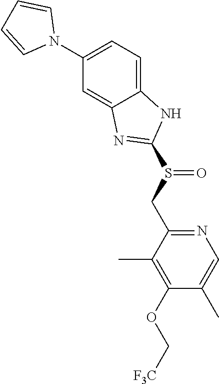 | 142) |
| 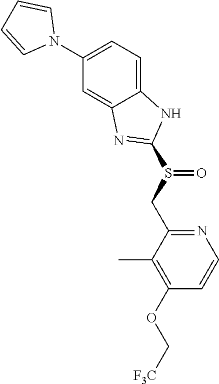 | 143) |
| 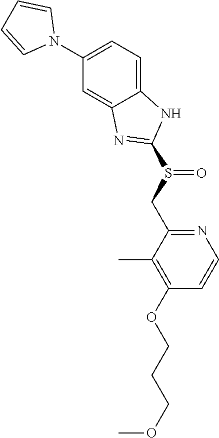 | 144) |
| 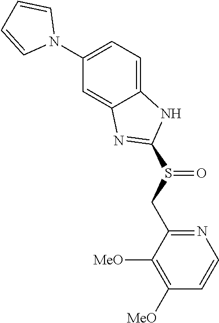 | 145) |
| 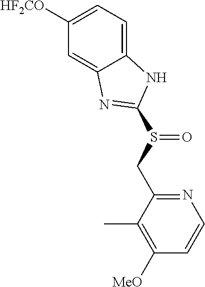 | 146) |
| 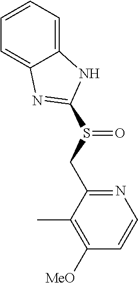 | 147) |
| 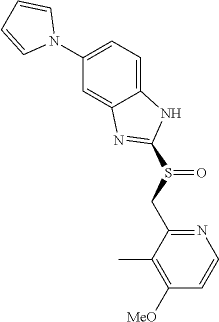 | 148) |
| 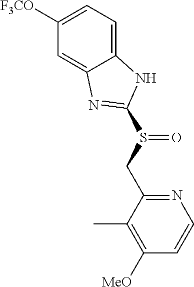 | 149) |
| 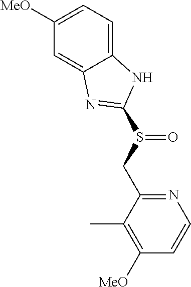 | 150) |

TABLE 6
Structure

| | |
|---|---|
| 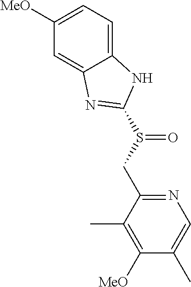 | 151) |
| 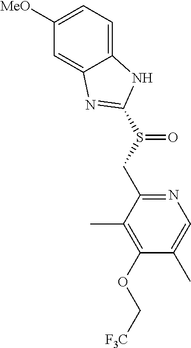 | 152) |
| 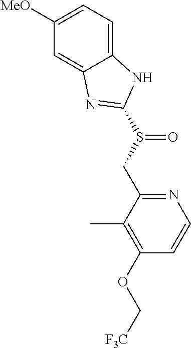 | 153) |
| 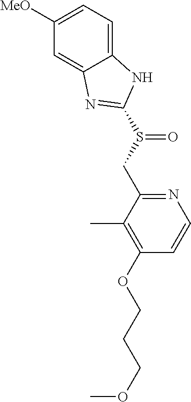 | 154) |
| 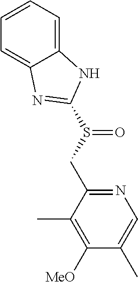 | 155) |
| 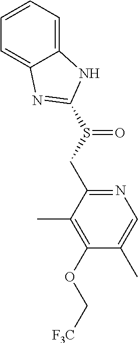 | 156) |
| 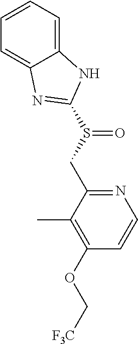 | 157) |
| 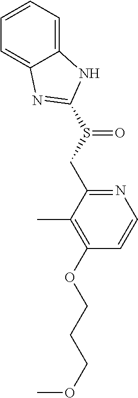 | 158) |
| 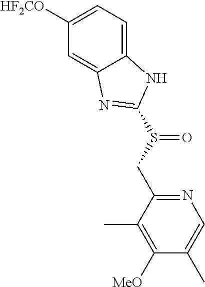 | 159) |
| 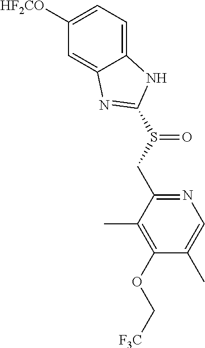 | 160) |
| 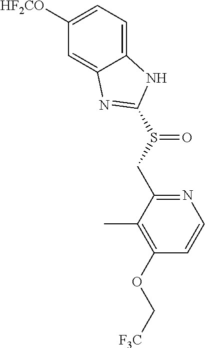 | 161) |
| 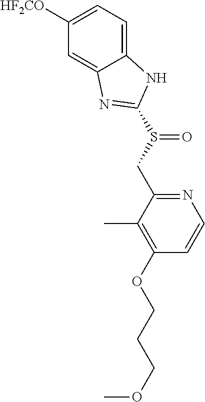 | 162) |
| 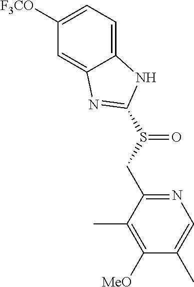 | 163) |
| 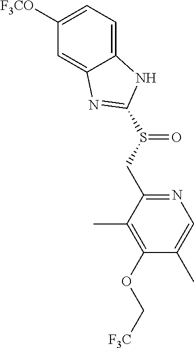 | 164) |
| 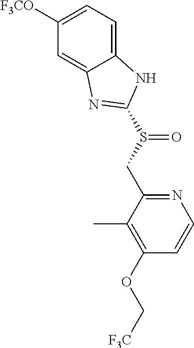 | 165) |
| 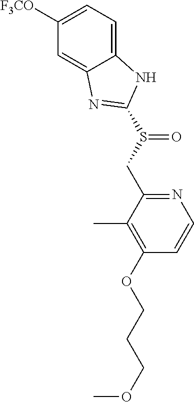 | 166) |
| 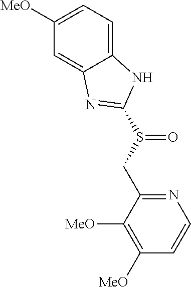 | 167) |
| 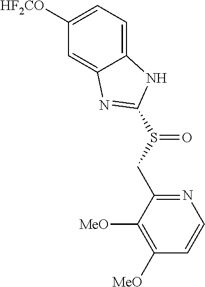 | 168) |
| 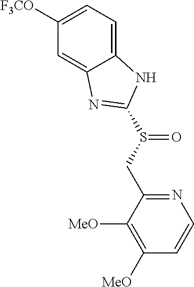 | 169) |
| 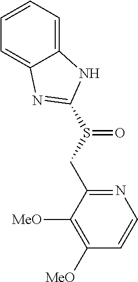 | 170) |
| 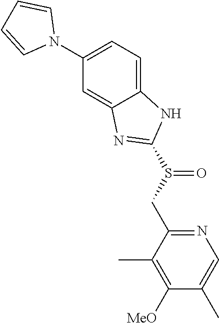 | 171) |
| 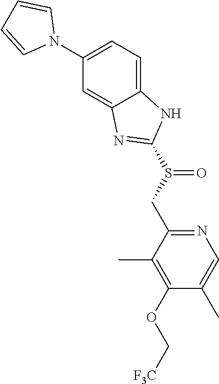 | 172) |
| 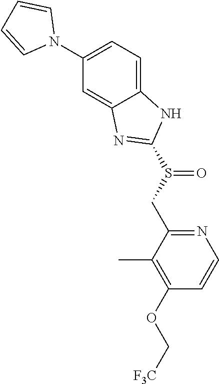 | 173) |
| 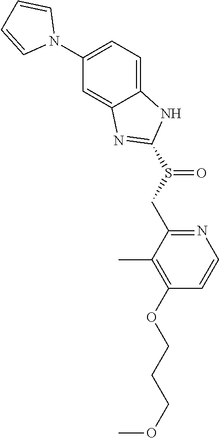 | 174) |
| 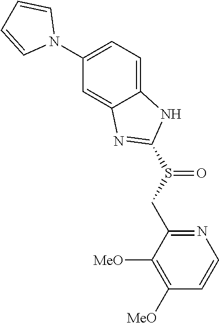 | 175) |
| 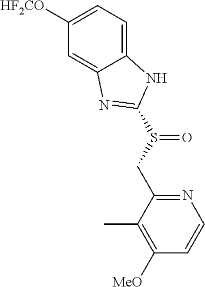 | 176) |
| 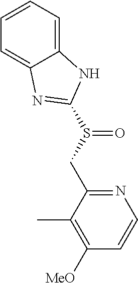 | 177) |
| 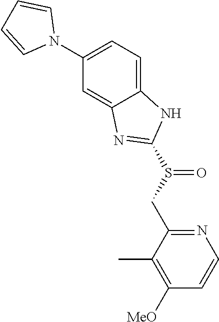 | 178) |
| 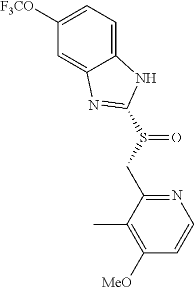 | 179) |
| 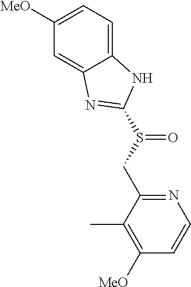 | 180) |

TABLE 7
Structure

| | |
|---|---|
| 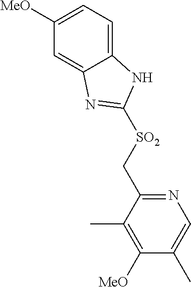 [SMILES: COc1ccc2[nH]c(S(=O)(=O)Cc3ncc(C)c(OC)c3C)nc2c1] | 181) |
| 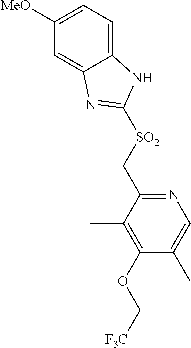 [SMILES: COc1ccc2[nH]c(S(=O)(=O)Cc3ncc(C)c(OCC(F)(F)F)c3C)nc2c1] | 182) |
| 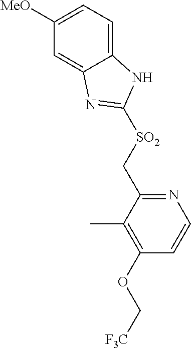 [SMILES: COc1ccc2[nH]c(S(=O)(=O)Cc3nccc(OCC(F)(F)F)c3C)nc2c1] | 183) |
| 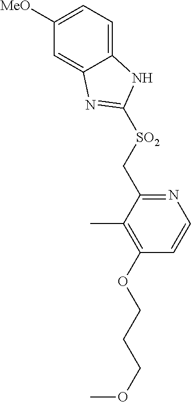 [SMILES: COCCCOc1ccnc(CS(=O)(=O)c2nc3cc(OC)ccc3[nH]2)c1C] | 184) |
| 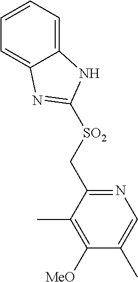 [SMILES: COc1c(C)cnc(CS(=O)(=O)c2nc3ccccc3[nH]2)c1C] | 185) |
| 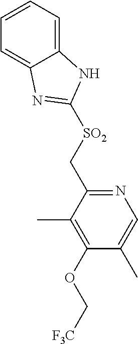 [SMILES: Cc1cnc(CS(=O)(=O)c2nc3ccccc3[nH]2)c(C)c1OCC(F)(F)F] | 186) |
| 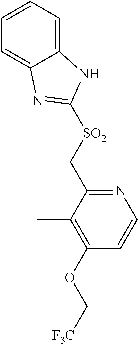 [SMILES: Cc1c(OCC(F)(F)F)ccnc1CS(=O)(=O)c1nc2ccccc2[nH]1] | 187) |
| 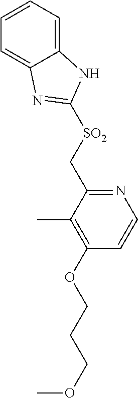 [SMILES: COCCCOc1ccnc(CS(=O)(=O)c2nc3ccccc3[nH]2)c1C] | 188) |
| 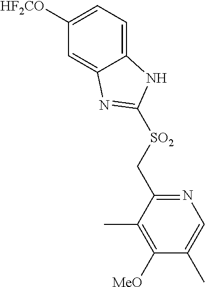 | 189) |
| 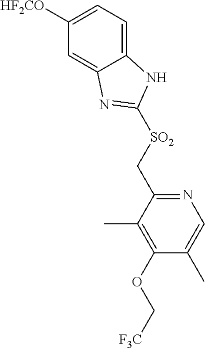 | 190) |
| 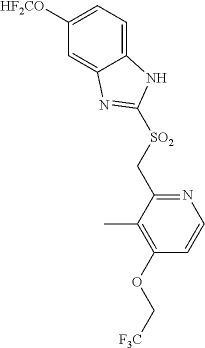 | 191) |
| 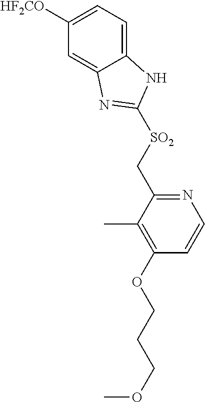 | 192) |
| 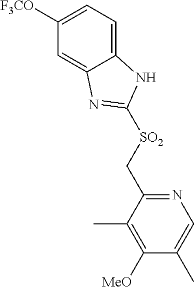 [SMILES: COc1c(C)cnc(CS(=O)(=O)c2nc3cc(OC(F)(F)F)ccc3[nH]2)c1C] | 193) |
| 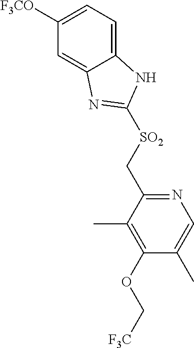 [SMILES: Cc1cnc(CS(=O)(=O)c2nc3cc(OC(F)(F)F)ccc3[nH]2)c(C)c1OCC(F)(F)F] | 194) |
| 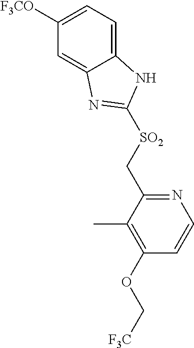 [SMILES: Cc1c(OCC(F)(F)F)ccnc1CS(=O)(=O)c1nc2cc(OC(F)(F)F)ccc2[nH]1] | 195) |
| 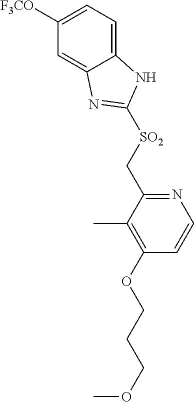 [SMILES: COCCCOc1ccnc(CS(=O)(=O)c2nc3cc(OC(F)(F)F)ccc3[nH]2)c1C] | 196) |
| 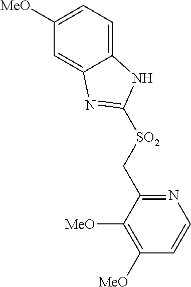 [SMILES: COc1ccc2[nH]c(S(=O)(=O)Cc3nccc(OC)c3OC)nc2c1] | 197) |
| 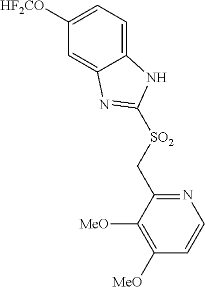 | 198) |
| 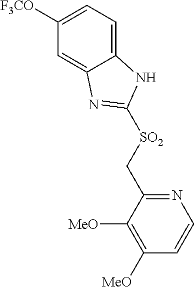 [SMILES: COc1ccnc(CS(=O)(=O)c2nc3cc(OC(F)(F)F)ccc3[nH]2)c1OC] | 199) |
| 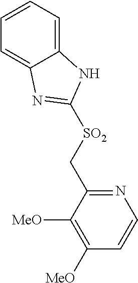 [SMILES: COc1ccnc(CS(=O)(=O)c2nc3ccccc3[nH]2)c1OC] | 200) |
| 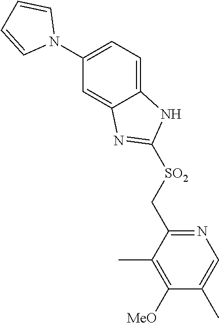 [SMILES: COc1c(C)cnc(CS(=O)(=O)c2nc3cc(-n4cccc4)ccc3[nH]2)c1C] | 201) |
| 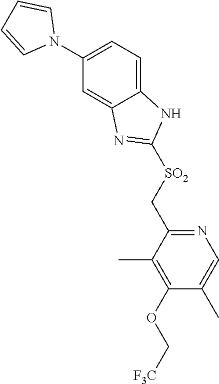 [SMILES: Cc1cnc(CS(=O)(=O)c2nc3cc(-n4cccc4)ccc3[nH]2)c(C)c1OCC(F)(F)F] | 202) |
| 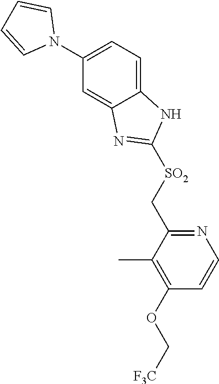 [SMILES: Cc1c(OCC(F)(F)F)ccnc1CS(=O)(=O)c1nc2cc(-n3cccc3)ccc2[nH]1] | 203) |
| 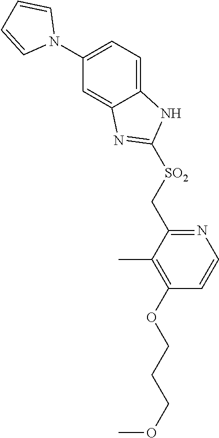 [SMILES: COCCCOc1ccnc(CS(=O)(=O)c2nc3cc(-n4cccc4)ccc3[nH]2)c1C] | 204) |
| 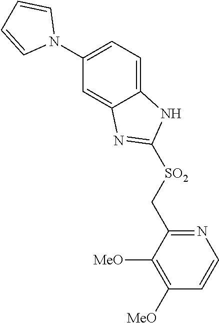 [SMILES: COc1ccnc(CS(=O)(=O)c2nc3cc(-n4cccc4)ccc3[nH]2)c1OC] | 205) |
| 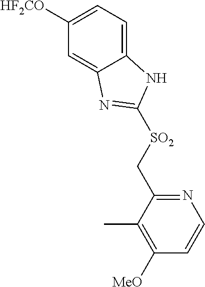 | 206) |
| 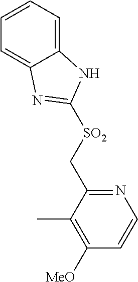 [SMILES: COc1ccnc(CS(=O)(=O)c2nc3ccccc3[nH]2)c1C] | 207) |
| 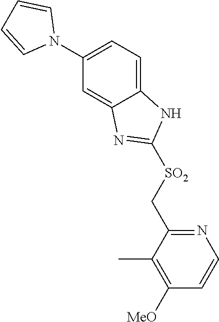 [SMILES: COc1ccnc(CS(=O)(=O)c2nc3cc(-n4cccc4)ccc3[nH]2)c1C] | 208) |
| 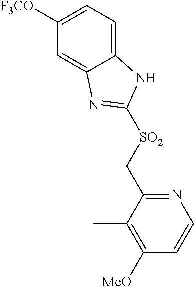 [SMILES: COc1ccnc(CS(=O)(=O)c2nc3cc(OC(F)(F)F)ccc3[nH]2)c1C] | 209) |
| 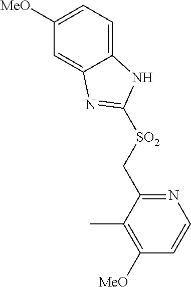 [SMILES: COc1ccc2[nH]c(S(=O)(=O)Cc3nccc(OC)c3C)nc2c1] | 210) |

### Methods of Inhibition of CXCL10 Production by Cells, Tissues and Organs.

The compounds and compositions described herein inhibit the gene expression and production of CXCL10 by various types of mammalian cells, tissues and organs. Non-limiting examples are inhibition of production of CCXL10 by keratinocytes, bronchial epithelial cells, peripheral blood monocyte cells, full thickness epidermal tissue and ocular retina under conditions known to increase CXCL10 gene expression and subsequent release CXCL10 protein into the cellular or tissue milieu, as measured by various assays including immune based protein quantitation.

### Methods of Treatment

The production of CXCL10 is induced in several cell and tissue types during the course of many diseases and exhibits pleiotropic effects on a wide range of biological processes including immunity, angiogenesis and organ-specific metastasis of cancers. Increased levels of CXCL10 in cells, tissues and blood have been shown to contribute directly to disease pathogenesis and progression. Production of CXCL10 has been shown to be directly contributory to the pathogenesis diseases affecting many organs systems.

This disclosure provides methods for treating CXCL10 mediated diseases or conditions selected from the group consisting of: Acquired Immunodeficiency Syndrome, Acute Kidney Injury, Acute Respiratory Distress Syndrome, Alzheimer Disease, Arthritis, Asthma, Astrocytoma, Behcet Syndrome, Bone Marrow Diseases, Breast Neoplasms, Bronchitis, Bronchopulmonary Dysplasia, Candidiasis, Ductal Breast Carcinoma, Hepatocellular Carcinoma, Non-Small-Cell Lung, Renal Cell Carcinoma, Chorea, Chorioamnionitis, Colitis, Colonic Neoplasms, Colorectal Neoplasms, Corneal Neovascularization Coronary Disease, Crohn Disease, Cryoglobulinemia, Cystic Fibrosis, Dementia, Dengue, Dermatitis, Diabetes Mellitus, Diabetes Mellitus, Dry Eye Syndromes, Eczema, Encephalitis, Endometrial Neoplasms, Endometriosis, Epstein-Barr Virus Infections, Fatigue, Fibrosis, Glioma, Gliosis, Granuloma, Graves' Disease, HIV Infections, Hepatitis, Idiopathic Pulmonary Fibrosis, Inflammatory Bowel Diseases, Kidney Neoplasms, Lichen Planus, Liver Cirrhosis, Lupus Erythematosus, Lupus Nephritis, Lymphoma, Macular Degeneration, Malaria, Melanoma, Malignant Melanoma, Multiple Sclerosis, Myasthenia Gravis, Mycoplasma Pneumonia, Myositis, Osteoarthritis, Pancreatitis, Parkinson Disease, Periodontal Disease, Polyradiculoneuropathy, Prader-Willi Syndrome, Pre-Eclampsia, Prostatic Neoplasms, Pruritus, Psoriasis, Pulmonary Emphysema, Pyelonephritis, Retinopathy, Sarcoidosis, Scleroderma, Sinusitis, Systemic Sepsis, Sjögren's Syndrome, Spondylitis, Still's Disease, Stomach Neoplasms, Stroke, Thyroiditis, Traumatic Brain Injury, Tuberculosis, Uterine Cervical Neoplasms, Uveitis, Wound degeneration.

An embodiment of this invention directed to a method of treating CXCL10 mediated diseases in a patient in need of such treatment comprising administering to the patient a therapeutically effective amount of at least one compound of formula (1), or a pharmaceutically acceptable salt or solvate thereof. The methods of treatment of this disclosure are especially advantageous in treating diseases where CXCL10 binds to CXCR3 receptor but are not limited to that binding occurring at physiological pH.

One embodiment of this invention is directed to a pharmaceutical composition comprising at least one compound of Formula 1, or a pharmaceutically acceptable salt or solvate thereof, in combination with a pharmaceutically acceptable carrier at physiological pH.

A preferred embodiment on this invention is directed to a topical route of administration of a pharmaceutical composition comprising at least one compound of formula (1), or a pharmaceutically acceptable salt or solvate thereof, in combination with a pharmaceutically acceptable carrier at physiological pH.

Another preferred embodiment of this disclosure is a method of treating a CXCL10 mediated disease by parenteral administration of (a) a therapeutically effective amount of at least one compound of formula (1), or a pharmaceutically acceptable salt or solvate thereof, in combination with a pharmaceutically acceptable carrier at physiological pH.

In an embodiment of the present invention the compounds according to Formula 1 (or formula 2) may be used either simultaneously or sequentially in combination with a second compound such as, but not limited to: non-steroidal anti-inflammatory drugs, such as aspirin, choline salicylate, celecoxib, acetaminophen, diclofenac, flurbiprofen, ibuprofen, indomethacin, ketoprofen, meclofenamate, mefenamic acid, nabumetone, naproxen, piroxicam, rofecoxib, salicylates, sulindac, tolmetin, and valdecoxib; immunomodulatory agents, such as methotrexate, azathioprine, mitoxantrone, cladribin, cyclophosphamide, tacrorimus, methotrexate, cyclosporine, and hydroxychloroquine; antimalarials, such as chloroquine, quinine, amodiaquine, pyrimethamine, proguanil, mefloquine, atovaquone, primaquine, artemisinin, and halofantrine; antibiotics, such as sulfonamides, clindamycin, members of the tetracycline family (including minocycline and doxycycline), erythromycin, and dapsone; anti-TNF alpha agents, such as infliximab, adalimumab, certolizumab pegol, golimumab, thalidomide, lenalidomide, pomalidomide, and etanercept; anti-CD20 agents, such as but not limited to: rituximab, obinutuzumab, Ibritumomab tiuxetan, and tositumomab; antidiarrheals, such as lidamidine, diphenoxylate, loperamide, and quercetin; antidepressants, such as amitriptyline, clomipramine, doxepin nortriptyline, and trimipramine; antipsychotics, such as droperidol, pimozide, chlorpromazine, thiothixene, loxapine, molindone, quetiapine, risperidone, sertindole, and zotepine; antifungals, such as clotimazole, flucisoconazole, abafungin, micafugin, terbinafine, ciclopirox, and tolnaftate; antihelminthics, such as mebendazole, levamisole, abamectin, and suramine; T lymphocyte activation inhibitors, such as cyclosporine, voclosporin, peroxynitrite, and dasatinib; anti-IL-1 agents, such as anakinra and IL-1Ra; antihyperglycemic agents, such as insulin, glyburide, glipizide, pioglitazone, acarbose, nateglinide, and metformin; glucocorticoids, such as methyl prednisolone, prednisolone, dexamethasone, betamethasone, fluticasone propionate, budesonide, flunisolide, mometasone furoate, triamcinolone acetonide, rofleponide, ciclesonide, and butixocort propionate; anti-cytokine/chemokine monoclonal antibodies, such as basiliximab, daclizumab, and secukinumab; sex steroids and receptor modulators, such as progesterone, progestins, androgen, estrogen, mifepristone, and misoprostil; antiacid agents, such as cimetidine, magnesium hydroxide, esomeprazole and calcium carbonate; anti-cellular surface receptor monoclonal antibodies directed against cell surface receptors such as CCR1, CCR3, CXCR2, CXCR3, CCL4, CCR5, IL7Ra, and TSLPR; aminosalicylic acid derivatives such as sulfasalazine and mesalazine; anticholinergic agents, such as ipratropium, oxitropium, tiotropium, dextromethorphan, revatropate, pirenzepine, darifenacin, oxybutynin, mecamylamine, terodiline, tolterodine, trospium chloride, and solifenacin; adrenergic agonists, such as but not limited to: salmeterol, salbutamol, clonidine, oxymetazoline, and dolbutamine; cholinergic agonists, such as carbachol, epibatidine, galantamine, nicotine, and varenicline; corticosteroids, such as cortisone and hydrocortisone; antineoplastic chemotherapeutic agents, such as cisplatin cyclophosphamide, bleomycin, doxorubicin, etoposide, folinic acid, and vincristine; phosphodiesterase inhibitors, such as mesembrenone, rolipram, Ibudilast, piclamilast, luteolin, drotaverine, roflumilast, cilomilast, apremilast, and crisaborole; leukotriene pathway modulators, such as 3-[3-butylsulfanyl-1-[(4-chlorophenyl)methyl]-5-propan-2-yl-indol-2-yl]-2,2-dimethyl-propanoic acid, baicalein, caffeic acid, curcumin, hyperforin, and zileuton; monoclonal antibodies directed against human immunoglobulins, such as omalizumab; adrenergic antagonists, such as alfluosin, idazoxan, labetalol, phentolamine, trazadone, propranolol, and atenolol; calcium channel antagonists, such as amelodipine, nifedapine, verapamil, diltiazem, and mibefradil; dopamine agonists, such as aripiprazole, bromocriptine, bupropion, cabergoline, lisuride, and roxindole; serotonin agonists, such as cabergoline, cisapride, gepirone, lorcaserin, and naratriptan; dopamine antagonists, such as amoxipine, bromopride, butaclamol, eticlopride, olanzapine, tiapride, and ziprasidone; serotonin antagonists, such as cyproheptadine, ketanserin, metergoline, methdilazine, oxetorone, and tropisetron; monoamine reuptake inhibitors, such as amineptine, citalopram, edivoxetine, hyperforin, mazindol, and viloxazine; protease inhibitors, such as amastatin, bestatin, and gabexate; histamine receptor antagonists, such as acrivastine, brompheniramine, cetirizine, cimetidine, ciproxifan, clobenprobit, cyclizine, carebastine, cyproheptadine, ebastine, epinastine, efletirizine, fexofenadine, and thioperamide; anti hypertriglycerides, such as fenofibrate, fibric acid, rosuvastatin, gemfibrozil, and omega-3-acid ethyl esters; HMG-CoA reductase inhibitors, such as atorvastatin, fluastatin, lovastatin, and simvastatin; retinoids such as etretinate, tretinoin, retinol, retinyl palmitate. So as to inhibit the biological activity of CXCL10 or otherwise aid in the treatment of the CXCL10 mediated disease or condition undergoing treatment.

### Pharmaceutical Compositions

Administration of the therapeutic agent may be by any suitable means. In some embodiments, the one or more therapeutic agents are administered by oral administration. In some embodiments, the one or more therapeutic agents are administered by transdermal administration. In some embodiments, the one or more therapeutic agents are administered by injection or intravenous infusion. In one embodiment, the one or more therapeutic agents, at physiological pH, are administered topically to a mucosal, dermal, or ocular tissue. In another embodiment, administration of the compounds disclosed herein can be by drug eluting devises or matrices so as provide a prolonged dosage of the compound over time.

If combinations of agents are administered as separate compositions, they may be administered by the same route or by different routes. If combinations of agents are administered in a single composition, they may be administered by any suitable route. In some embodiments, combinations of agents are administered as a single composition by oral administration. In some embodiments, combinations of agents are administered as a single composition by transdermal administration. In some embodiments, the combinations of agent are administered as a single composition by injection. In some embodiments, the combinations of agent are administered as a single composition topically.

In one embodiment of the present invention the compounds of Formula 1 may contain asymmetric or chiral centers and, therefore, exist in different stereoisomeric forms. For example, 2,2,2-trifluoroethyl 2-(4-methylphenoxy)propanoate is a compound according Formula 1 that possesses a chiral center at carbon atom number 8 and thus has two stereoisomer forms. It is intended that all stereoisomeric forms of the compounds of Formula 1 form part of the present invention, including but not limited to: diastereomers, enantiomers, and atropisomers as well as mixtures thereof, such as racemic mixtures. In addition, the present invention embraces all geometric and positional isomers. For example, if a compound of Formula 1 incorporates a double bond or a fused ring, both the cis- and trans-forms, as well as mixtures, are embraced within the scope of the invention. Both the single positional isomers and mixture of positional isomers are also within the scope of the present invention.

In one embodiment of the present invention, compounds of Formula 1 may exist in different tautomeric forms, and all such forms are embraced within the scope of the invention, as defined by the claims.

The dose and dosing regimens of the compound present in the invention may be adjusted to provide the optimum desired response in accordance with methods and practices well known in the therapeutic arts. For example, a single bolus dose may be administered, or several divided doses may be administered over time. The dose may also be proportionally reduced or increased as indicated by the exigencies of the therapeutic situation. The appropriate dosing regimen, the amount of each dose administered and/or the intervals between doses will depend upon a number of factors, including: the compound, the type of pharmaceutical composition, the characteristics of the subject in need of treatment and the severity of the condition being treated.

The dose of the compound will vary, but as a general guideline for dermatological administration, the compound will be present in a dermatologically acceptable formulation in a therapeutically effective dose in an amount of from about 0.0001 mg/kg to about 1000 mg/kg/body weight per day of a compound provided herein. The pharmaceutical compositions therefore should provide a dosage of from about 0.0001 mg/kg/body weight to about 1000 mg/kg/body weight of the compound for some conditions. In another embodiment of the present invention the pharmaceutical dosage unit forms are prepared to provide a preparation for topical application containing 0.01 to 50 w/w %, and more typically from about 0.1 to 10 w/w %. In yet other embodiments the pharmaceutical dosage unit forms are prepared to provide a preparation for topical application containing from 0.01% to 30% (w/v) of the compounds.

In some embodiments, the formulation may be applied to the affected area from 1 to 6 times daily. A “dermatologically acceptable formulation” is one that may be applied to the skin or hair and will allow the drug to diffuse to the site of action.

In some embodiment the compounds can be formulated into topically applied eye drops or gels using suitable salts or solvates thereof in combination with a pharmaceutically acceptable carrier. Preferred specific pharmaceutical carriers are known to those skilled in the art that facilitate penetration of the compounds to all regions of the eye such as but not limited to the: anterior segment, posterior segment, sclera, choroid and retina.

In some embodiments the compounds can be formulated into solid or semi-solid matrices that serve to provide a reservoir of the compound within a tissue or organ so as to provide a continuous supply of the compound for treatment over days, weeks or months.

The skilled artisan can also be expected to readily determine the maximum tolerable dose, the therapeutically effective amount which provides a detectable therapeutic benefit to a patient, and the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

The determination of optimal dosages for a particular patient is well-known to those skilled in the art. Certain non-limiting examples of pharmaceutically acceptable vehicles suitable for topical administration include propylene glycol:transcutanol:ethanol (20:20:60, v/v/v) and propylene glycol:ethanol (30:70, v/v). In some embodiments, the compound of Formula 1 may be present at concentrations of between about 0.1% to about 10% (w/v).

In another embodiment, the medicinal and cosmetic formulations containing the compound and any additional therapeutic agents will typically be packaged for retail distribution (i.e. an article of manufacture or a kit). Such articles will be labeled and packaged in a manner to instruct the patient how to use the product. Such instructions will include the condition to be treated, duration of treatment, dosing schedule, etc. The compound(s) of Formula 1 may also be admixed with any inert carrier and utilized in laboratory assays in order determine the concentration of the compounds within the serum, urine, etc., of the patient as is known in the art. The compound may also be used as a research tool.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention belongs. The following examples and biological data are being presented in order to further illustrate the invention. This disclosure should not be construed as limiting the invention in any manner.

For all of the foregoing embodiments, each embodiment disclosed herein is contemplated as being applicable to each of the other disclosed embodiments. Those skilled in the art will readily appreciate that the specific Experimental Details which follow are only illustrative of the invention as described more fully in the claims which follow thereafter.

### Examples

### 1. Inhibition of CXCL10 Gene Expression and CXC110 Protein Production in Human Cells and Tissues

To determine the ability of compounds, according to Formula 1 to inhibit CXCL10 gene expression, protein production and secretion using functional assays we used cultivated normal human keratinocytes, bronchial epithelial cells, human peripheral blood mononuclear cells and reconstituted full thickness human epithelium tissue. These cells and tissue were treated with stimulants known to induce gene expression, production and secretion of CXCL10. Various compounds of the invention were tested for their ability to inhibit CXCL10 gene expression and/or production of the CXCL10 protein. The gene expression and production of CXCL10 protein were determined by well-known methods as described below.

Human keratinocyte cells (NEHK) from healthy volunteers were obtained and cultivated at 1×106 cells/ml using 6 well plates in NEHK-GM media according to supplier's protocol (MatTek Corporation; Ashland, Mass.). Human bronchial epithelial cells (NHBE) were obtained and cultivated at 1×106 cells/ml using 6 well plates in NHBE-GM media according to supplier's protocol (MatTek Corporation; Ashland, Mass.). Human peripheral blood monocytic cells (PBMC) were obtained by Ficol density gradient centrifugation and cultivated at 1×106 cells/ml using 6 well plates in cultured at 1×106 cells/ml in RPMI-1640 medium (GIBCO® Inc. Carlsbad, Calif., USA) supplemented with 20% fetal bovine serum and 1% streptomycin/penicillin according to standard protocol (ThermoFisher-Invitrogen; Carlsbad Calif.).

The EpiDermFT (EFT-400) a reconstructed full thickness human epithelial tissue, (MatTek Corporation; Ashland, Mass.) was used to evaluate CXCL10 gene expression and CXCL10 protein production and secretion from intact human tissue. The tissues were cultivated and matured according to manufacturer's protocol. After maturation of the EpiDermFT tissues was achieved, the tissues were then used for testing.

Human cell suspensions and tissues without stimulation agents known to induce CXCL10 production were used as a baseline control for the experiments. Neither the unstimulated cells nor the tissues expressed the CXCL10 gene or produced CXCL10 protein in measurable amounts. The cell cultures and tissues were treated with various exemplar compounds according to Formula 1 at several concentrations for 8 hours prior to stimulation by CXCL10 inducing agents. The tissue samples were also tested with various exemplar compounds according to Formula 1 by applying the test compound to the apical surface of the tissue for 8 hours prior to stimulation by CXCL10 inducing agents.

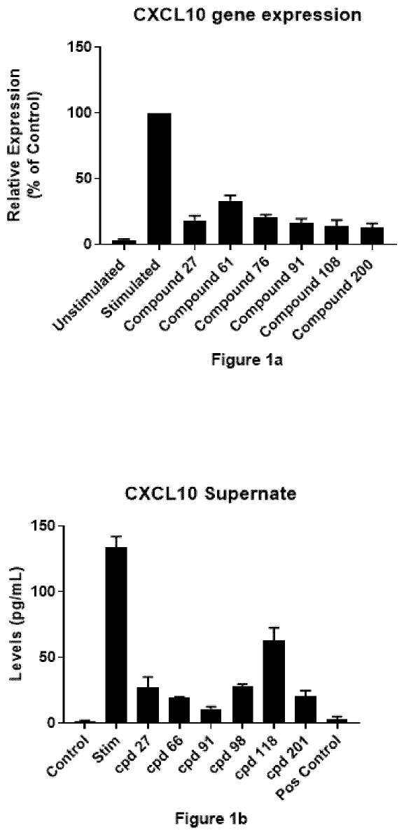

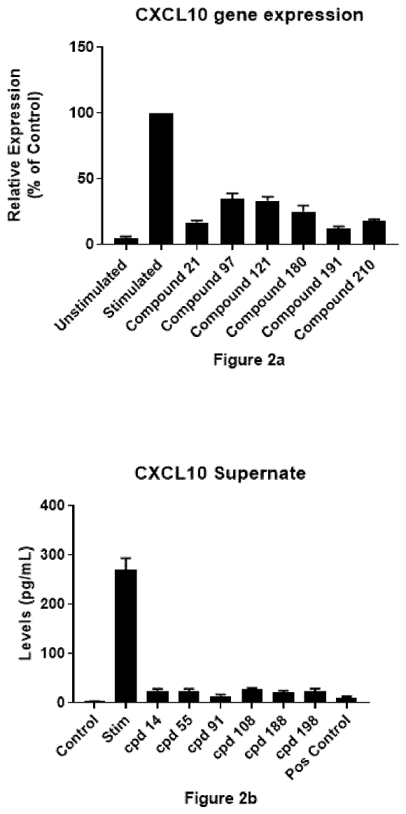

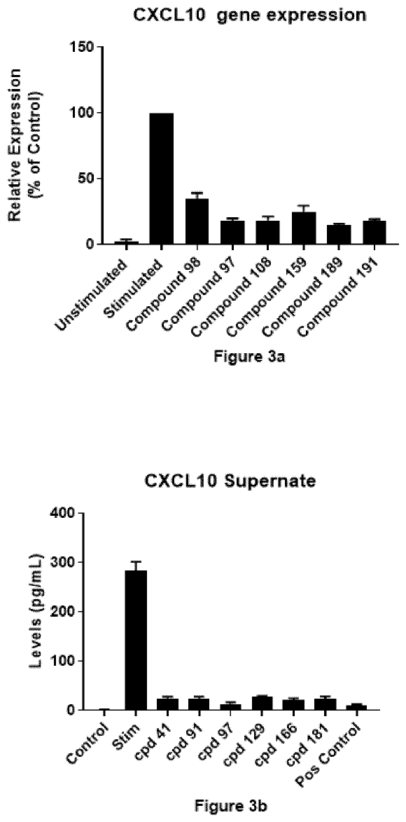

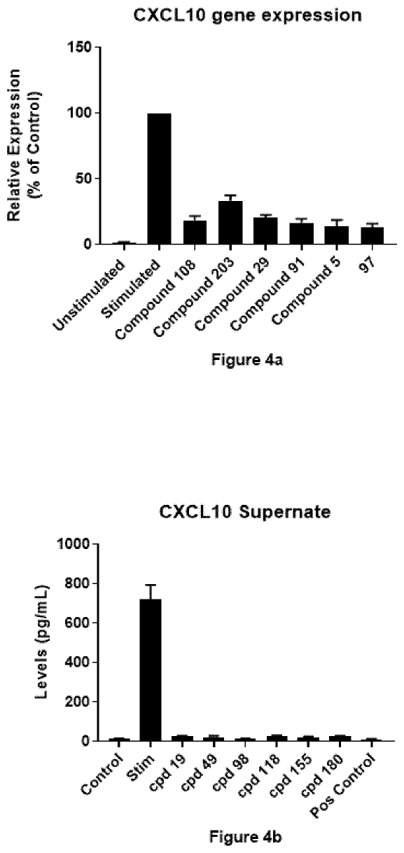

The cell cultures of NHEK, NHBE, and PBMC were stimulated with various known stimulators of CXCL10 production that consisted of: Poly [I:C] 10 ug/ml (Invivogen; San Diego, Calif.) (see FIG. 1a and FIG. 1b), Lipopolysaccharide (LPS) 10 ug/ml (Invivogen; San Diego, Calif.) (see FIGS. 2a and 2b), Interferon gamma (IFN-γ) 10 ng/ml (Invivogen; San Diego, Calif.) (see FIGS. 3a and 3b). The EFT-400 tissues were stimulated with Interferon gamma (IFN-γ) 10 ng/ml plus Tumor Necrosis Factor alpha (TNF-α) 10 ng/ml (Invivogen; San Diego, Calif.) which act synergistically to maximally induce CXCL10 (see FIGS. 4a and 4b).

Measurement of CXCL10 gene expression was determined from total RNA extracted from cell pellets and tissues after incubation by RNAseq quantitation method standardized using expression of housekeeping genes as reference standard. CXCL10 protein production was measured in the cell and tissue culture supernatants after incubation using the CXCL10 ELISA kit (R&D Systems; Minneapolis, Minn.). The results of these assays demonstrated that induction of CXCL10 gene expression, subsequent protein expression and secretion was significantly inhibited by the compounds.

### 2. In Vivo Inhibition of CXCL10 Gene Expression and Associated Retinopathy in Rats

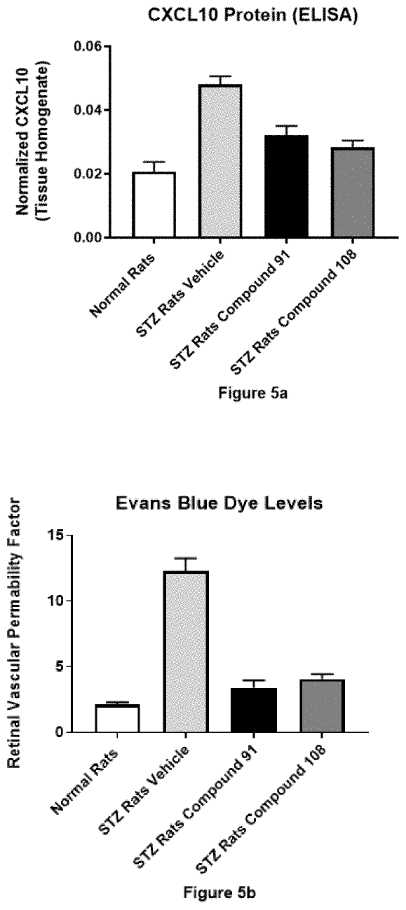

CXCL10 expression is associated with age related and diabetes induced retinopathy. A hallmark of these retinopathies is retinal vascular leakage (i.e. increased retinal vascular permeability) thought to induced by CXCL10. To test the in vivo effect of the compounds on treatment of retinopathies associated with increased CXCL10 levels, diabetes was induced in rats by treatment with Streptozotocin (STZ) according established methods (Akbarzadeh et al, J Clin Biochem. 2007 September; 22(2): 60-64) Testing began once the diabetes had been established in the animals. Compounds were formulated into a topically applied eye drop solution containing 1-5% of compounds 91 and 108 along with ethylene glycol, sodium lauryl sulfate, hyaluronic acid, and sterile water at pH 7.0. The drops containing the test compound were applied (20 μL) three times per day in one eye and drops that did not contain the test compound were applied to the other eye in similar fashion for 30 days. CXCL10 expression was measured in retina tissue homogenate and retinal vascular permeability was assayed using the measurement of leakage of Evans Blue dye from retinal vessels, a well-established method (Invest Ophthalmol Vis Sci 2001 March; 42: 789-794). The treatment of the animals with the compounds demonstrated a significant reduction in the amount of CXCL10 produced by the retina and a reduction of the retinal vascular permeability as compared to the control eye (FIG. 5a-b).

### 3. Clinical Treatment of Dermatitis/Eczema

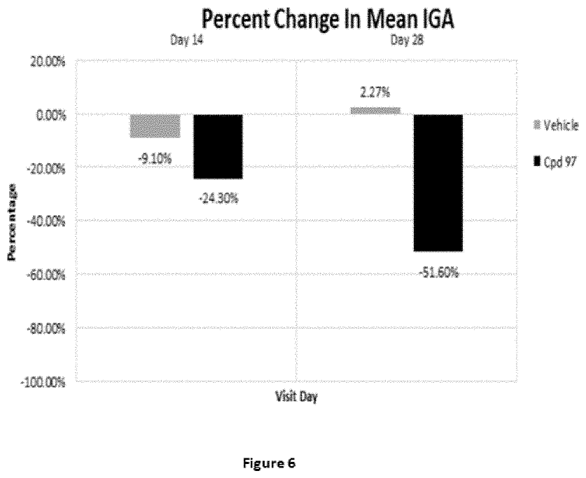

To test the effect of the compounds on treatment of human dermatologic disorders associated with increased CXCL10 production by keratinocytes, Compound 97 and Compound 98 were formulated with stearic acid, lanolin (anhydrous), mineral oil and triethanolamine into a topically applied cream at a concentration of 7%. The compounds in the cream base were applied topically twice per day to human subjects suffering from dermatitis in an area of the skin with active skin lesions for 3-4 weeks. The control was the topical cream base composition without the active compounds. Outcome was measured by an experienced clinical observer as assessed by visible reduction or disappearance of the lesions as assessed in the treated area vs the control area treated only with the cream base alone using Investigator's Global Assessment (IGA) scale. The study revealed the compounds had a pronounced effect on reducing the dermatitis/eczema (FIG. 6).

### 4. In Vivo Inhibition of CXCL10 Expression and Uvetitis

CXCL10 expression is associated with ocular inflammation (uveitis). Therefore, to test the effect of the compounds to treat uveitis, C57BL/6 mice were induced to have experimental autoimmune uveitis (EAU) using standard methods Caspi R R et al. Journal of immunology (Baltimore, Md. 1988; 140: 1490-1495). Briefly, EAU was induced by active immunization with bovine interphotoreceptor retinoid-binding protein (IRBP), using 150 ug in a 0.2 ml emulsion (1:1 v/v with complete Freund's adjuvant (CFA) containing Mycobacterium tuberculosis strain H37RA (2.5 mg/mL). EAU is an established preclinical animal model for assessment of immunotherapeutic efficacy treatments for posterior uveitis. The course and severity of the induced ocular inflammation can be evaluated and objectively scored using Topical Endoscope Fundus Imaging (TEFI) as described by Paques et al. (Invest Ophthalmol Vis Sci. 2007; 48:2769-2774) and modified by Copland et al. (Invest Ophthalmol Vis Sci. 2008; 49:5458-5465). The time course and severity of the induced uveitis in the mice (treated vs vehicle control) was followed over a 3-week period and TEFI was measured. Experiment was performed in triplicate for treatment verses negative control vehicle only. The study revealed that the compounds tested had a pronounced effect on reducing the EAU.

The results of the experiments and clinical tests herein demonstrate that compounds according to Formula 1 inhibit the production of CXCL10 from human cells and tissues. In addition, they demonstrate a clinical therapeutic effect in subjects suffering from dermatologic disorder and in an established animal model of a retinal disorder associated with production of CXCL10. Therefore, the compounds according to Formula 1 are useful in to reduce the production of CXCL10 in human cells and tissues induced by various stimulants and for treatment of various disorders associated with increased CXCL10 production such as but not limited to dermatitis and retinopathy.

### LIST OF REFERENCES

D. J. Abraham et al. (2010) “Burger's Medicinal Chemistry, Drug Discovery and Development” Wiley, 7th Edition
T. L. Lemke et al. (2013) “Foye's Principles of Medicinal Chemistry” Wiley, 7th Edition
M. B. Smith (2013) “March's Advanced Organic Chemistry: Reactions, Mechanisms, and Structure” Wiley, 7th Edition
S. Hanessian et al. (2013) “Design and Strategy in Organic Synthesis” Wiley-VCH, 1st Edition
P. G. M. Wuts et al. “Greene's Protective Groups in Organic Synthesis” Wiley-Interscience, 4th Edition
T. L. Ho “Fiesers' Reagents for Organic Synthesis” Wiley, Volume 27 Edition
Kohl B, Sturn E, Senn-Bilfinger J et al, J. Med. Chem, 35, 1049-1053 (1992)
Lindberg P L, Von Unge S, U.S. Pat. No. 5,714,504 (1998)
Cotton H et al, WO 9854171 (1998)

1. A method of treating a disorder associated with increased expression of CXCL10 in a human subject comprising administering to a patient in need thereof an effective amount of a composition comprising a pharmaceutically acceptable carrier and a compound,
pharmaceutically acceptable salt, ester, or prodrug of Formula 1 at physiological pH 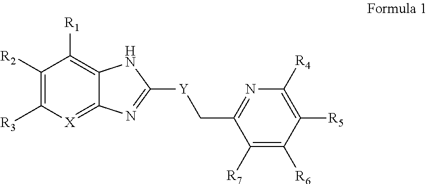
wherein:
X is selected from CH or N;
Y is selected from CH₂, CHOH, C(optionally substituted C₁ to C₈ straight chain or branched chain alkyl)OH, C═O, NH, N-optionally substituted C₁ to C₈ straight chain or branched chain alkyl, NCO-optionally substituted C₁ to C₈ straight chain or branched chain alkyl, S, S═O, SO₂;
R₁, R₂, R₃, R₄, R₅, R₆, and R₇ are independently selected from:
H; OH; F; Cl; Br; I; (halogen)alkyl, optionally substituted C₁ to C₈ straight chain or branched chain alkyl; optionally substituted C₁ to C₈ cycloalkyl; heterocycloalkyl; alkylheterocycloalkyl; optionally substituted C₁ to C₈ alkenyl; optionally substituted C₁ to C₈ alkynyl; optionally substituted aryl; optionally substituted alkylaryl; optionally substituted heteroaryl; optionally substituted alkylheteroaryl; O-alkyl; O-optionally substituted alkyl, O-cycloalkyl; O-alkylcycloalkyl; O-aryl; O-optionally substituted aryl; alkyl-O-aryl; alkyl-O-optionally substituted aryl; C(O)-aryl; C(O)-optionally substituted aryl; CH₂C(O)-aryl; CH₂C(O)-optionally substituted aryl; O-(halogen)alkyl, wherein optionally substituted alkenyl, if present, may have one or more double bond and each double bond may independently be cis or trans, E or Z, a cis/trans mixture or an E/Z mixture, or
adjacent substituents R₁ and R₂, R₂ and R₃, R₄ and R₅, R₅ and R₆, R₆ and R₇ may form a saturated or unsaturated 5 membered or 6-membered or 7 membered carbocyclic or heterocyclic ring,
wherein if at least one asymmetric center is present the compound may be in the form of a racemic mixture, a single enantiomer, a diastereoisomeric mixture, an enantiomeric diastereomer, a meso compound, a pure epimer, or a mixture of epimers thereof,
and wherein a hydrogen, several hydrogens or all hydrogens may be replaced with deuterium, or a pharmaceutically acceptable salt, ester or prodrug form thereof.

2. The method according to claim 1, wherein the human disorder is selected from any one of: Acquired Immunodeficiency Syndrome, Acute Kidney Injury, Alzheimer Disease, Arthritis, Asthma, Astrocytoma, Behcet Syndrome, Breast Neoplasms, Bronchitis, Chronic Obstructive Pulmonary Disease, Crohn Disease, Cryoglobulinemia, Cystic Fibrosis, Dermatitis, Diabetic Retinopathy, Dry Eye Syndrome, Encephalitis, Endometriosis, Fibrosis, Gliosis, Hepatitis, Hepatocellular Carcinoma, Idiopathic Pulmonary Fibrosis, Interstitial Cystitis, Kidney Neoplasm, Lichen Planus, Liver Biliary Cirrhosis, Lupus Erythematosus, Lupus Nephritis, Lymphocytic Choriomeningitis, Macular Degeneration, Melanoma, Multiple Sclerosis, Myasthenia Gravis, Myositis, Non-Small-Cell Carcinoma of the Lung, Non-Renal Cell Carcinoma, Osteoarthritis, Pancreatitis, Parkinson Disease, Polyradiculoneuropathy, Prader-Willi Syndrome, Pre-Eclampsia, Psoriasis, Renal Cell Carcinoma, Retinopathy of Prematurity, Sarcoidosis, Scleroderma, Sjogren's Syndrome, Spondylitis, Ulcerative Colitis, Uveitis, or Wound degeneration.

3. The method according to claim 2, wherein the human disorder is Diabetic Retinopathy.

4. The method according to claim 2, wherein the human disorder is uveitis.

5. The method according to claim 2, wherein the human disorder is macular degeneration.

6. The method according to claim 2, wherein the human disorder is dermatitis.

7. The method according to claim 2, wherein the human disorder is interstitial cystitis.

8. The method of claim 1, wherein the compound according to claim 1 is administered simultaneously or sequentially in combination with other compounds such as, but not limited to: non-steroidal anti-inflammatory drugs; immunomodulatory agents; antimalarials; antibiotics; anti-TNF alpha agents; anti-CD20 agents; antidiarrheals; Bioactive peptides; corticosteroids; antidepressants; antipsychotics; antifungals; antihelminthics; T lymphocyte activation inhibitors; anti-IL-1 agents; antihyperglycemic agents; glucocorticoids; anti-cytokine/chemokine monoclonal antibodies; sex steroids and receptor modulators; antiacid agents; anti-cellular surface receptor monoclonal antibodies directed against cell surface receptors; aminosalicylic acid derivatives; adrenergic agonists; cholinergic agonists; corticosteroids; antineoplastic chemotherapeutic agents; phosphodiesterase inhibitors; leukotriene pathway modulators; monoclonal antibodies directed against human immunoglobulins; adrenergic antagonists; calcium channel antagonists; dopamine agonists; serotonin agonists; dopamine antagonists; serotonin antagonists; monoamine reuptake inhibitors; protease inhibitors; histamine receptor antagonists; anti hypertriglycerides; HMG-CoA reductase inhibitors; and retinoids.

9. A compound according to Formula 1 below: 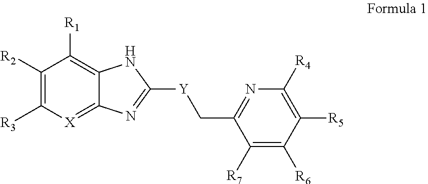
wherein:
X is selected from CH or N;
Y is selected from CH₂, CHOH, C(optionally substituted C₁ to C₈ straight chain or branched chain alkyl)OH, C═O, NH, N-optionally substituted C₁ to C₈ straight chain or branched chain alkyl, NCO-optionally substituted C₁ to C₈ straight chain or branched chain alkyl, S, S═O, SO₂;
R₁, R₂, R₃, R₄, R₅, R₆, and R₇ are independently selected from: H; OH; F; Cl; Br; I; (halogen)alkyl, optionally substituted C₁ to C₈ straight chain or branched chain alkyl; optionally substituted C₁ to C₈ cycloalkyl; heterocycloalkyl; alkylheterocycloalkyl; optionally substituted C₁ to C₈ alkenyl; optionally substituted C₁ to C₈ alkynyl; optionally substituted aryl; optionally substituted alkylaryl; optionally substituted heteroaryl; optionally substituted alkylheteroaryl; O-alkyl; O-optionally substituted alkyl, O-cycloalkyl; O-alkylcycloalkyl; O-aryl; O-optionally substituted aryl; alkyl-O-aryl; alkyl-O-optionally substituted aryl; C(O)-aryl; C(O)-optionally substituted aryl; CH₂C(O)-aryl; CH₂C(O)-optionally substituted aryl; O-(halogen)alkyl, or adjacent substituents R₁ and R₂, R₂ and R₃, R₄ and R₅, R₅ and R₆, R₆ and R₇, may form a saturated or unsaturated 5 membered or 6-membered or 7 membered carbocyclic or heterocyclic ring, and optionally substituted alkenyl, if present, may have one or more double bonds and each double bond may independently be cis or trans, E or Z, a cis/trans mixture or an E/Z mixture, wherein if at least one asymmetric center is present the compound may be in the form of a racemic mixture, a single enantiomer, a diastereoisomeric mixture, an enantiomeric diastereomer, a meso compound, a pure epimer, or a mixture of epimers thereof, and a hydrogen, several hydrogens or all hydrogens may be replaced with deuterium;
or a pharmaceutically acceptable salt, ester or prodrug form thereof.

10. The compound according to claim 9, wherein the compound is selected from the group consisting of compounds 1-30.

11. The compound according to claim 9, wherein the compound is selected from the group consisting of compounds 31-60.

12. The compound according to claim 9, wherein the compound is selected from the group consisting of compounds 61-90.

13. The compound according to claim 9, wherein the compound is selected from the group consisting of compounds 91-120.

14. The compound according to claim 9, wherein the compound is selected from the group consisting of compounds 121-150.

15. The compound according to claim 9, wherein the compound is selected from the group consisting of compounds 151-180.

16. The compound according to claim 9, wherein the compound is selected from the group consisting of compounds 181-210.

17. A pharmaceutical composition comprising a compound of claim 9 or a tautomer thereof and a pharmaceutically acceptable vehicle, diluent and/or carrier.

18. A pharmaceutical combination comprising a therapeutically effective amount of a composition comprising:
(a) a first compound according to claim 9; and
(b) a second compound selected from the group consisting of Non-steroidal anti-inflammatory drugs, Immunomodulatory agents, Anti-malarials, Antibiotics, Anti-TNF alpha agents, Anti-CD20 agents, Anti-diarrheal drugs, Antidepressants, Anti-psychotics, Anti-fungals, Anti-hypertriglycerides, Anti-helminthics, T lymphocyte activation inhibitors, Anti-IL-1 agents, Cholinergic agonists, Glucocorticoids, Anti-cytokine/chemokine monoclonal antibodies, Sex steroids and receptor modulators, Anti-cellular surface receptor monoclonal antibodies, Aminosalicylic acid derivatives, Anticholinergic agents, Adrenergic agonists, Corticosteroids, Anti-neoplastic chemotherapeutic agents, Phosphodiesterase inhibitors, Leukotriene pathway modulators, Monoclonal antibodies directed against human immunoglobulins, Adrenergic antagonists, Calcium channel antagonists, Dopamine agonists, Serotonin agonists, Dopamine antagonists, Serotonin antagonists, Monoamine reuptake inhibitors, Protease inhibitors, Histamine receptor antagonists, Retinoids, and HMG-CoA reductase inhibitors.

19. A method of inhibiting CXCL10 gene expression and/or protein production in a mammal comprising administering to a mammal an effective amount of a compound according to claim 9.

20. A method of inhibiting CXCL10 secretion in a mammal comprising administering to a mammal an effective amount of a compound according to claim 9.
